(19) United States
(12) Patent Application Publication
(10) Pub. No.: US 2018/0177872 A1
(43) Pub. Date: Jun. 28, 2018

(54) Combination of PD-1 antagonist with an EGFR inhibitor

(71) Applicants: Yong Jia, San Diego, CA (US); Shailaja Kasibhatla, San Diego, CA (US); Sanela Bilic, Urbandale, IA (US); John Cameron, Belmont, MA (US); Danny Howard, JR., Washington, NJ (US)

(72) Inventors: Yong Jia, San Diego, CA (US); Shailaja Kasibhatla, San Diego, CA (US); Sanela Bilic, Urbandale, IA (US); John Cameron, Belmont, MA (US); Danny Howard, JR., Washington, NJ (US)

(21) Appl. No.: 15/877,436

(22) PCT Filed: Jul. 27, 2016
(86) PCT No.: PCT/IB2016/054488

§ 371 Date: Jan. 23, 2018

Related U.S. Application Data
(60) Provisional application No. 62/331,371, filed on May 3, 2016.
(60) Provisional application No. 62/198,390, filed on Jul. 29, 2015.

(51) Int. Cl. A61K 39/395; A61K 47/68; C07K 16/28; A61P 35/00

(52) U.S. Cl. CPC A61K 39/39558; A61P 35/00; C07K 16/2818; A61K 47/6803

(57) ABSTRACT

The present invention relates to a pharmaceutical composition comprising PD-1 antagonist and an EGFR Inhibitor. The present combination can be administered independently or separately, in a quantity which is jointly therapeutically effective for the treatment of cancer. The invention also provides the use of such a combination for the manufacture of a medicament; the use of such a combination as a medicine; a kit of part comprising such a combination; and a method of treatment of such a combination.

### SEQUENCE LISTING

The instant application contains a Sequence Listing which has been submitted electronically in ASCII format and is hereby incorporated by reference in its entirety. Said ASCII copy, created on Jul. 25, 2016, is named PAT057001_SL.txt and is 64,034 bytes in size.

### FIELD OF THE DISCLOSURE

The present invention relates to a pharmaceutical composition comprising a PD-1 antagonist and an EGFR inhibitor. The present combination is administered independently or separately, in a quantity which is jointly therapeutically effective for lung cancer, e.g. squamous lung cancer and NSCLC, colorectal cancer and breast cancer, e.g., triple-negative breast cancer (TNBC). The invention further relates to a use of such a combination for the manufacture of a medicament; the use of such combination as a medicine; a kit of parts comprising such a combination; a dosing regimen using the combination disclosed herein, and a method of treatment of lung cancer, e.g. squamous lung cancer and NSCLC, colorectal cancer and breast cancer, e.g., triple-negative breast cancer (TNBC), involving the combination.

### BACKGROUND OF THE DISCLOSURE

Lung cancer is the most common cancer worldwide, with NSCLC accounting for approximately 85% of lung cancer cases. In Western countries, 10-15% non-small cell lung cancer (NSCLC) patients express epidermal growth factor receptor (EGFR) mutations in their tumors and Asian countries have reported rates as high as 30-40%. The predominant oncogenic EGFR mutations (L858R and ex19del) account for about 90% of EGFR NSCLC.

Besides the classic EGFR mutations (L858R and Ex19Del), EGFR Exon 20 insertion mutations (Ex20ins) were described to account for 4-10% of all EGFR mutations in patients, the third largest EGFR mutant patient population behind the classic (L858R and ex19del) EGFR mutations.

EGFR-mutant patients are given an EFGR inhibitor as first line therapy. However, most patients develop acquired resistance, generally within 10 to 14 months. In up to 50% of NSCLC patients harboring a primary EGFR mutation treated with first generation reversible EGFR Tyrosine Kinase Inhibitors (TKIs) such as erlotinib and gefitinib, a secondary “gatekeeper” T790M mutation develops.

Second-generation EGFR TKIs (such as afatinib and dacomitinib) have been developed to try to overcome this mechanism of resistance. These are irreversible agents that covalently bind to cysteine 797 at the EGFR ATP site and are potent on both activating [L858R, ex19del] and acquired T790M mutations in pre-clinical models. Their clinical efficacy has however proven to be limited, possibly due to severe adverse effects caused by concomitant wild-type (WT) EGFR inhibition.

This has led to the development of third-generation EGFR TKIs which are WT EGFR sparing and also have relative equal potency for activating EGFR mutations [L858R, ex19del] and acquired T790M. Third generation EFGR TKIs such as AZD9291 (mereletinib) and CO-1686 (rociletinib) are thus beginning to enter clinical development and to show significant initial promise (e.g., see “AZD9291 in EGFR Inhibitor-Resistant Non-Small-Cell Lung Cancer”, Hanne et al, N Engl J Med, 2015; 372; 1689-99 and “Rociletinib in EGFR-Mutated Non-Small-Cell Lung Cancer”, Sequist et al, J Med, 2015; 372; 1700-9). See also “ASP8273, a novel mutant-selective irreversible EGFR inhibitor, inhibits growth of non-small cell lung cancer (NSCLC) cells with EGFR activating and T790M resistance mutations “Sakagami et al, AACR; Cancer Res 2014; 74; 1728.

Treatment with EGFR inhibitors has however not been shown to definitively translate into prolonged overall survival.

Agents that enhance anti-tumor immunity have recently been developed for the treatment of cancer. However, these treatments are not effective in all cancer types, responses are often not durable, and many patients receive little or no benefit from treatment. For instance, the activity of PD-1 inhibitors in lung cancer, and in particular, NSCLC, has so far been limited to a minority of patients. There is thus the need to develop further treatment options for cancer which is resistant or refractory to immunotherapy such as anti-PD-1 or anti-PD-L1 therapy. Examples of such therapies include therapy with pembrolizumab, nivolumab, atezolizumab and MEDI4736.

Breast cancer is the second most common cancer in the world with approximately 1.7 million new cases in 2012 and the fifth most common cause of death from cancer, with approximately 521,000 deaths. Of these cases, approximately 15% are triple-negative, which do not express the estrogen receptor, progesterone receptor (PR) or HER2. As such, these patients do not benefit from targeted therapies available to patients with other breast cancer subtypes. Triple-negative breast cancer (TNBC) is an aggressive disease and outcomes after therapy are poor.

Colorectal cancer (CRC) is the third most common cancer in the world, with approximately 1.4 million people diagnosed in 2012, and the fourth most common cause of death from cancer, with 694,000 deaths. Outcomes for patients with CRC are linked to the immune infiltrate in tumors, suggesting CRC may benefit from therapies that stimulate an immune response However, preliminary experience with checkpoint inhibitors of programmed death-1 (PD-1) have been disappointing outside of the mismatch repair-deficient population. The reasons for lack of efficacy are unclear.

Hence there is still a need for more effective treatment options for patients with lung cancer, such as squamous lung cancer and NSCLC, colorectal cancer and breast cancer, and in particular, triple-negative breast cancer (TNBC). There is also a need for treatment options for cancers, such as lung cancer, such as squamous lung cancer and NSCLC, colorectal cancer and breast cancer, and in particular, triple-negative breast cancer (TNBC), which prove to be resistant or refractory to immunotherapy such as anti-PD-1 or anti-PD-L1 therapy.

### SUMMARY OF THE DISCLOSURE

The TEC-family protein tyrosine kinases ITK, RLK and TEC have been identified as key components of T-cell-receptor signaling that contributes to the regulation and polarization of T-cell activation. Functional studies have implicated TEC kinases as important mediators of pathways that control CD4+T helper cell differentiation and promote effector functions. ITK is specific for T cells and is critically required for Th2 differentiation. TEC kinases have now emerged as important modulators of T-cell function that have exciting therapeutic potential for the regulation of polarized T-cell responses.

Compound A, i.e. (R,E)-N-(7-chloro-1-(1-(4-(dimethylamino)but-2-enoyl)azepan-3-yl)-1Hbenzo [d]imidazol-2-yl)-2-methylisonicotinamide, has been found to have immunomodulatory function due to its cross-reactivity on the TEC family of kinases, in particular ITK. ITK is expressed and regulates Th2 cell differentiation. Inhibition of TEC kinases and particularly ITK could shift the balance from Th2 to Th1 cells. Skewing the microenvironment from a Th2 to Th1 with Compound A may improve the antitumor immune response in some patients, particularly in combination with other immune modulators such as the antibody molecules disclosed herein.

The present invention therefore provides a novel combination of an EGFR inhibitor and a Programmed Death 1 (PD-1) antagonist that can provide an advantageous effect for treatment of specific cancers. The present invention therefore provides therapies which provide safe, effective treatment for patients suffering from cancer. It is also important that the patients continue to respond positively to such treatment for as long as possible. The combination of an EGFR inhibitor and a Programmed Death 1 (PD-1) antagonist may be particularly useful in the treatment of lung cancer, such as squamous lung cancer and NSCLC, colorectal cancer and breast cancer, and in particular, triple-negative breast cancer (TNBC).

The invention provides a pharmaceutical combination including an isolated antibody molecule capable of binding to a human Programmed Death-1 (PD-1) antagonist comprising (a) a heavy chain variable region (VH) comprising a HCDR1, a HCDR2 and a HCDR3 amino acid sequence of BAP049-Clone-B or BAP049-Clone-E as described in Table 1 and a light chain variable region (VL) comprising a LCDR1, a LCDR2 and a LCDR3 amino acid sequence of BAP049-Clone-B or BAP049-Clone-E as described in Table 1; and ii) (R,E)-N-(7-chloro-1-(1-(4-(dimethylamino)but-2-enoyl)azepan-3-yl)-1Hbenzo[d]imidazol-2-yl)-2-methylisonicotinamide (Compound A), or a pharmaceutically acceptable salt thereof. A useful salt of Compound A is the hydrochloride salt or the mesylate salt thereof. Compound A may also be in the free form (i.e. not a salt).

The invention also provides the pharmaceutical combination described above for use in the treatment of a cancer, such as lung cancer (e.g. squamous lung cancer and NSCLC), colorectal cancer and breast cancer (in particular, triple-negative breast cancer (TNBC)), which prove to be resistant, relapsing, or refractory to immunotherapy such as anti-PD-1 or anti-PD-L1 therapy. Examples of these therapies include therapy with pembrolizumab, nivolumab, atezolizumab and MEDI4736.

The pharmaceutical combination described herein includes a quantity which is therapeutically effective for the treatment of a cancer such as lung cancer, such as squamous lung cancer and NSCLC, colorectal cancer and breast cancer, and in particular, triple-negative breast cancer (TNBC), which prove to be resistant or refractory to immunotherapy such as anti-PD-1 or anti-PD-L1 therapy. Examples of these therapies include therapy with pembrolizumab, nivolumab, atezolizumab and MEDI4736.

In another aspect, the invention includes the use of the pharmaceutical combination described herein for the manufacture of a medicament for the treatment of a cancer such as lung cancer, such as squamous lung cancer and NSCLC; colorectal cancer and breast cancer, (and in particular, triple-negative breast cancer (TNBC)), which prove to be resistant or refractory to immunotherapy such as anti-PD-1 or anti-PD-L1 therapy. Examples of these therapies include therapy with pembrolizumab, nivolumab, atezolizumab and MEDI4736.

Novel dosage regimens involving the pharmaceutical combinations described herein are also provided.

The anti-PD-1 antibody molecule, e.g. BAP049-Clone-B or BAP049-Clone-E, is preferably administered or used at a flat or fixed dose.

Accordingly, in one aspect, the invention features a method of treating the cancers described herein wherein the method includes administering to the subject a pharmaceutical combination described herein wherein the anti-PD-1 antibody molecule, e.g. BAP049-Clone-B or BAP049-Clone-E, is administered at a dose of about 300 mg to 400 mg once every three weeks or once every four weeks. In certain embodiments, the anti-PD-1 antibody molecule, e.g. BAP049-Clone-B or BAP049-Clone-E, is administered at a dose of about 300 mg once every three weeks. In other embodiments, the anti-PD-1 antibody molecule, e.g. BAP049-Clone-B or BAP049-Clone-E, is preferably administered at a dose of about 400 mg once every four weeks.

### BRIEF DESCRIPTION OF THE FIGURES

FIG. 1 depicts the predicted Ctrough (Cmin) concentrations across the different weights for patients while receiving the same dose of an exemplary anti-PD-1 antibody molecule. A comparison of predicted mean steady state C_{trough} after body weight versus flat/fixed dosing of two regimens of the anti-PD-1 antibody molecule is shown.

FIG. 2 depicts observed versus model predicted (population or individual based) Cmin concentrations.

FIG. 3 depicts the accumulation, time course and within subject variability of the model used to analyze pharmacokinetics. The shaded areas represent 90% prediction interval; solid lines are the median of prediction at each time point; black dots represent observed pharmacokinetic data.

FIG. 4 depicts the percent survival of mice bearing A20 lymphoma allografts after treatment with Compound A (also known as EGF816), ibrutinib, anti-PD-L1 antibody, a combination of Compound A (EGF816) and anti-PD-L1 antibody, or a combination of ibrutinib and anti-PD-L1 antibody.

FIG. 5 depicts the mean tumor volume in mice bearing A20 lymphoma allografts after treatment with Compound A (also known as EGF816), ibrutinib, anti-PD-L1 antibody, a combination of EGF816 and anti-PD-L1 antibody, or a combination of ibrutinib and anti-PD-L1 antibody. Bar indicates treatment period for EGF816 and ibrutinib. Arrows indicate when anti-PD-L1antibody was administered.

### SPECIFIC DESCRIPTION OF THE DISCLOSURE

There exists a need for therapeutically effective combinations that can be used to treat cancer, in particular solid tumors. The present invention is directed to a combination of Compound A and an anti-PD-1 antibody as shown in Table 1 that can be used to treat cancers. While not wishing to be bound by theory the use of the novel combination disclosed herein to treat a particular cancer is believed to be advantageous as it affects the immune response rescuing T cell antitumor response and expanding the endogenous antitumor response of T cells. After activation, T cells increase the expression of PD-1 on their surface, allowing them to receive a negative signal thereby inhibiting T cell responses. Tumor cells have taken advantage of this system by expressing binding partners of PD-1, such as PD-L1, that prematurely shut down T cell responses against the tumor. In the present combination, the anti-PD 1 antibody molecule recognizes and binds PD-1 on T cells thereby preventing the tumor cells from binding PD-1 and reducing T cell activity. The anti-PD-1 antibody molecule binds the T cell but does not interfere with T cell function thus ensuring that T cells retain their tumor killing affect.

Compound A is a targeted covalent irreversible EGFR inhibitor that selectively inhibits activating and acquired resistance mutants (L858R, ex19del and T790M), while sparing WT EGFR. (see Jia et al, Cancer Res Oct. 1, 2014 74; 1734). Compound A has shown significant efficacy in EGFR mutant (L858R, ex19del and T790M) cancer models (in vitro and in vivo) with no indication of WT EGFR inhibition at clinically relevant efficacious concentrations.

Compound A demonstrated strong tumor regressions in several EGFR activating and resistant tumor models in vivo. These include HCC827 (ex19del), H3255 (L858R) and H1975 (L858R; T790M) that are representative of the relevant clinical settings. In all of the models Compound A inhibited tumor growth in a dose-dependent manner and achieved regressions of established tumors at well tolerated doses. Compound A is predicted to have improved antitumor activity in humans with known EGFR-driven cancers.

As discussed herein, Compound A was also found to have immune-modulatory potential. Compound A is thus expected to stimulate a more effective anti-tumor immune response. Enhancing the antitumor immune response is thus expected to be beneficial across the diseases described herein.

A combined small molecule targeted-immunotherapy approach may provide clinical benefit, such as improved and sustained therapy for patients suffering from cancer e.g, lung cancer, such as squamous lung cancer and NSCLC, colorectal cancer and breast cancer, and in particular, triple-negative breast cancer (TNBC); and also in lung cancer, such as squamous lung cancer and NSCLC, colorectal cancer and breast cancer, and in particular, triple-negative breast cancer (TNBC), which prove to be resistant or refractory to immunotherapy such as anti-PD-1 or anti-PD-L1 therapy. Examples of these therapies include therapy with pembrolizumab, nivolumab, atezolizumab and MEDI4736.

EGFR Inhibitor

The present invention relates to the use of (R,E)-N-(7-chloro-1-(1-(4-(dimethylamino)but-2-enoyl)azepan-3-yl)-1Hbenzo[d]imidazol-2-yl)-2-methylisonicotinamide (Compound A), or a pharmaceutically acceptable salt thereof. Compound A is also known as and herein referred by the code “EGF816”. A particularly useful salt of Compound A is the mesylate salt thereof. WO2013/184757, the contents of which are hereby incorporated by reference, describes Compound A, its method of preparation and pharmaceutical compositions comprising Compound A. Compound A has the following structure:

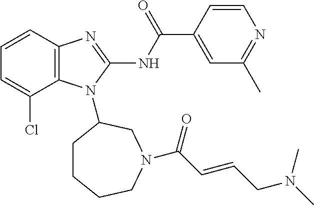 [SMILES: Cc1cc(C(=O)Nc2nc3cccc(Cl)c3n2C2CCCCN(C(=O)/C=C/CN(C)C)C2)ccn1]

Compound A may be in the free form (i.e. not a salt). Alternatively, Compound A may be present as a salt. Compound A may be present as the hydrocholoride salt or the mesylate (methylsulphonate) salt, more preferably as the mono-mesylate salt. Said mesylate salts may be in an amorphous or crystalline state. A particularly useful salt form of Compound A is the mono-mesylate trihydrate salt thereof. Free forms, salt forms and pharmaceutical compositions of Compound A are described in PCT application PCT/IB2014/066475, which published as WO/2015/083059.

Compound A also inhibits one or more kinases in the TEC family of kinases. The Tec family kinases include, e.g., ITK, BMX, TEC, RLK, and BTK, and are central in the propogation of T-cell receptor and chemokine receptor signaling (Schwartzberg et al. (2005) Nat. Rev. Immunol. p. 284-95). For example, Compound A can inhibit ITK with a biochemical IC50 of 1.3 nM. ITK is a critical enzyme for the survival of Th2 cells and its inhibition results in a shift in the balance between Th2 and Th1 cells. Combined treatment, in vivo, with the ITK inhibitor ibrutinib or Compound A, and anti-PD-L1 antibody results in superior efficacy compared with either single agent in several models.

The combination of ITK inhibition (with ibrutinib) and checkpoint inhibition is more effective than either single agent in numerous syngeneic mouse models, e.g., those which express ITK but not BTK. The synergistic effect of ITK inhibition and checkpoint blockade has been tested in mouse allografts using mouse cancer cell lines (A20, CT26 and 4T1) (Sagiv-Barfi et al. (2015) Blood. p. 2079-86). The combination of anti-PD-L1 antibody and ibrutinib (an ITK inhibitor) was shown to be significantly more efficacious than either single agent in all three models. In these experiments, the treatment effect was prolonged despite the dosing of ibrutinib for only 8 days, and a total of 5 doses of anti-PD-L1 antibody. Approximately half of the CT26 tumor bearing mice treated with this combination were cured (no mice treated with either single agent were cured). Rechallenge of these mice with CT26 tumor inoculum demonstrated long term anti-tumor memory specific for this cell line (Sagiv-Barfi et al. (2015) Blood. p. 2079-86). Furthermore, tumor specific T-cells were found in the blood and spleen of mice treated with ibrutinib and anti-PD-L1 antibody. A similar experiment was performed using Compound A in the A20 lymphoma model (see e.g., Example 4). The combination of either Compound A and anti-PD-L1 antibody or ibrutinib and anti-PD-L1antibody was more effective than a single agent. Compound A and ibrutinib were dosed for only ten days, and a total of 5 doses of anti-PD-L1 antibody were given. Compound A and ibrutinib were only dosed transiently and the effects of Compound A plus anti-PD-L1 antibody and ibrutinib plus anti-PD-L1 antibody on survival extended beyond 60 days. Combination of anti-PD-L1 antibody and Compound A resulted in tumor regression in mice bearing A20 lymphoma allografts. Accordingly, in some embodiments, the EGFR inhibitor, (R,E)-N-(7-chloro-1-(1-(4-(dimethylamino)but-2-enoyl)azepan-3-yl)-1H-benzo [d]imidazol-2-yl)-2-methylisonicotinamide (Compound A), or a pharmaceutically acceptable salt thereof, enhances, or is used to enhance an antitumor effect of an inhibitor of PD-1 (e.g., an anti-PD-1 antibody molecule).

In some embodiments, the EGFR inhibitor is chosen from one of more of erlotinib, gefitinib, cetuximab, panitumumab, necitumumab, PF-00299804, nimotuzumab, or RO5083945.

PD-1 Antagonists

The PD-1 molecules useful in the present invention are shown in Table 1 and are described in PCT application PCT/US2015/012754, which was published on 30 Jul. 2015, as WO/2015/112900, and which is incorporated herein in its entirety by reference.

In one embodiment, the anti-PD-1 antibody molecule is a humanized anti-PD-1 antibody and includes a heavy chain variable domain and a constant region, a light chain variable domain and a constant region, or both, comprising the amino acid sequence of BAP049-Clone-B or BAP049-Clone-E as described in Table 1, or encoded by the nucleotide sequence in Table 1. The anti-PD-1 antibody molecule, optionally, comprises a leader sequence from a heavy chain, a light chain, or both, as shown in Table 2; or a sequence substantially identical thereto.

In yet another embodiment, the anti-PD-1 antibody molecule includes at least one, two, or three complementarity determining regions (CDRs) from a heavy chain variable region of an antibody described herein, e.g., an antibody chosen from any of BAP049-Clone-B or BAP049-Clone-E as described in Table 1, or encoded by the nucleotide sequence in Table 1

In one embodiment, e.g., an embodiment comprising a variable region, a CDR (e.g., Chothia CDR or Kabat CDR), or other sequence referred to herein, e.g., in Table 1, the antibody molecule is a monospecific antibody molecule, a bispecific antibody molecule, or is an antibody molecule that comprises an antigen binding fragment of an antibody, e.g., a half antibody or antigen binding fragment of a half antibody.

TABLE 1
Heavy and light chains of BAP049-Clone-B and BAP049-Clone-E
including heavy and light variable domains and CDRs

| | | |
|---|---|---|
| BAP049-Clone-B HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 6 (Chothia) | HCDR3 | WTTGTGAY |
| SEQ ID NO: 7 | VH | EVQLVQSGAEVKKPGESLRISCKGSG |
| | | YTFTTYWMHWVRQATGQGLEWMG |
| | | NIYPGTGGSNFDEKFKNRVTITADKS |
| | | TSTAYMELSSLRSEDTAVYYCTRWT |
| | | TGTGAYWGQGTTVTVSS |
| SEQ ID NO: 8 | DNA VH | GAGGTGCAGCTGGTGCAGTCAGGC |
| | | GCCGAAGTGAAGAAGCCCGGCGAG |
| | | TCACTGAGAATTAGCTGTAAAGGT |
| | | TCAGGCTACACCTTCACTACCTACT |
| | | GGATGCACTGGGTCCGCCAGGCTA |
| | | CCGGTCAAGGCCTCGAGTGGATGG |
| | | GTAATATCTACCCCGGCACCGGCG |
| | | GCTCTAACTTCGACGAGAAGTTTA |
| | | AGAATAGAGTGACTATCACCGCCG |
| | | ATAAGTCTACTAGCACCGCCTATAT |
| | | GGAACTGTCTAGCCTGAGATCAGA |
| | | GGACACCGCCGTCTACTACTGCACT |
| | | AGGTGGACTACCGGCACAGGCGCC |
| | | TACTGGGGTCAAGGCACTACCGTG |
| | | ACCGTGTCTAGC |
| SEQ ID NO: 9 | HC | EVQLVQSGAEVKKPGESLRISCKGSG |
| | | YTFTTYWMHWVRQATGQGLEWMG |
| | | NIYPGTGGSNFDEKFKNRVTITADKS |
| | | TSTAYMELSSLRSEDTAVYYCTRWT |
| | | TGTGAYWGQGTTVTVSSASTKGPSV |
| | | FPLAPCSRSTSESTAALGCLVKDYFP |
| | | EPVTVSWNSGALTSGVHTFPAVLQS |
| | | SGLYSLSSVVTVPSSSLGTKTYTCNV |
| | | DHKPSNTKVDKRVESKYGPPCPPCP |
| | | APEFLGGPSVFLFPPKPKDTLMISRTP |
| | | EVTCVVVDVSQEDPEVQFNWYVDG |
| | | VEVHNAKTKPREEQFNSTYRVVSVL |
| | | TVLHQDWLNGKEYKCKVSNKGLPS |
| | | SIEKTISKAKGQPREPQVYTLPPSQEE |
| | | MTKNQVSLTCLVKGFYPSDIAVEWE |
| | | SNGQPENNYKTTPPVLDSDGSFFLYS |
| | | RLTVDKSRWQEGNVFSCSVMHEAL |
| | | HNHYTQKSLSLSLG |
| SEQ ID NO: 10 | DNA HC | GAGGTGCAGCTGGTGCAGTCAGGC |
| | | GCCGAAGTGAAGAAGCCCGGCGAG |
| | | TCACTGAGAATTAGCTGTAAAGGT |
| | | TCAGGCTACACCTTCACTACCTACT |
| | | GGATGCACTGGGTCCGCCAGGCTA |
| | | CCGGTCAAGGCCTCGAGTGGATGG |
| | | GTAATATCTACCCCGGCACCGGCG |
| | | GCTCTAACTTCGACGAGAAGTTTA |
| | | AGAATAGAGTGACTATCACCGCCG |
| | | ATAAGTCTACTAGCACCGCCTATAT |
| | | GGAACTGTCTAGCCTGAGATCAGA |
| | | GGACACCGCCGTCTACTACTGCACT |
| | | AGGTGGACTACCGGCACAGGCGCC |
| | | TACTGGGGTCAAGGCACTACCGTG |
| | | ACCGTGTCTAGCGCTAGCACTAAG |
| | | GGCCCGTCCGTGTTCCCCCTGGCAC |
| | | CTTGTAGCCGGAGCACTAGCGAAT |
| | | CCACCGCTGCCCTCGGCTGCCTGGT |
| | | CAAGGATTACTTCCCGGAGCCCGT |
| | | GACCGTGTCCTGGAACAGCGGAGC |
| | | CCTGACCTCCGGAGTGCACACCTTC |
| | | CCCGCTGTGCTGCAGAGCTCCGGG |
| | | CTGTACTCGCTGTCGTCGGTGGTCA |
| | | CGGTGCCTTCATCTAGCCTGGGTAC |
| | | CAAGACCTACACTTGCAACGTGGA |
| | | CCACAAGCCTTCCAACACTAAGGT |
| | | GGACAAGCGCGTCGAATCGAAGTA |
| | | CGGCCCACCGTGCCCGCCTTGTCCC |
| | | GCGCCGGAGTTCCTCGGCGGTCCCT |
| | | CGGTCTTTCTGTTCCCACCGAAGCC |
| | | CAAGGACACTTTGATGATTTCCCGC |
| | | ACCCCTGAAGTGACATGCGTGGTC |
| | | GTGGACGTGTCACAGGAAGATCCG |
| | | GAGGTGCAGTTCAATTGGTACGTG |
| | | GATGGCGTCGAGGTGCACAACGCC |
| | | AAAACCAAGCCGAGGGAGGAGCA |
| | | GTTCAACTCCACTTACCGCGTCGTG |
| | | TCCGTGCTGACGGTGCTGCATCAG |
| | | GACTGGCTGAACGGGAAGGAGTAC |
| | | AAGTGCAAAGTGTCCAACAAGGGA |
| | | CTTCCTAGCTCAATCGAAAAGACC |
| | | ATCTCGAAAGCCAAGGGACAGCCC |
| | | CGGGAACCCCAAGTGTATACCCTG |
| | | CCACCGAGCCAGGAAGAAATGACT |
| | | AAGAACCAAGTCTCATTGACTTGC |
| | | CTTGTGAAGGGCTTCTACCCATCGG |
| | | ATATCGCCGTGGAATGGGAGTCCA |
| | | ACGGCCAGCCGGAAAACAACTACA |
| | | AGACCACCCCTCCGGTGCTGGACT |
| | | CAGACGGATCCTTCTTCCTCTACTC |
| | | GCGGCTGACCGTGGATAAGAGCAG |
| | | ATGGCAGGAGGGAAATGTGTTCAG |
| | | CTGTTCTGTGATGCATGAAGCCCTG |
| | | CACAACCACTACACTCAGAAGTCC |
| | | CTGTCCCTCTCCCTGGGA |
| BAP049-Clone-B LC | | |
| SEQ ID NO: 11 (Kabat) | LCDR1 | KSSQSLLDSGNQKNFLT |
| SEQ ID NO: 12 (Kabat) | LCDR2 | WASTRES |
| SEQ ID NO: 13 (Kabat) | LCDR3 | QNDYSYPYT |
| SEQ ID NO: 14 (Chothia) | LCDR1 | SQSLLDSGNQKNF |
| SEQ ID NO: 15 (Chothia) | LCDR2 | WAS |
| SEQ ID NO: 16 (Chothia) | LCDR3 | DYSYPY |
| SEQ ID NO: 17 | VL | EIVLTQSPATLSLSPGERATLSCKSSQ |
| | | SLLDSGNQKNFLTWYQQKPGKAPKL |
| | | LIYWASTRESGVPSRFSGSGSGTDFT |
| | | FTISSLQPEDIATYYCQNDYSYPYTFG |
| | | QGTKVEIK |
| SEQ ID NO: 18 | DNA VL | GAGATCGTCCTGACTCAGTCACCC |
| | | GCTACCCTGAGCCTGAGCCCTGGC |
| | | GAGCGGGCTACACTGAGCTGTAAA |
| | | TCTAGTCAGTCACTGCTGGATAGCG |
| | | GTAATCAGAAGAACTTCCTGACCT |
| | | GGTATCAGCAGAAGCCCGGTAAAG |
| | | CCCCTAAGCTGCTGATCTACTGGGC |
| | | CTCTACTAGAGAATCAGGCGTGCC |
| | | CTCTAGGTTTAGCGGTAGCGGTAGT |
| | | GGCACCGACTTCACCTTCACTATCT |
| | | CTAGCCTGCAGCCCGAGGATATCG |
| | | CTACCTACTACTGTCAGAACGACTA |
| | | TAGCTACCCCTACACCTTCGGTCAA |
| | | GGCACTAAGGTCGAGATTAAG |
| SEQ ID NO: 19 | LC | EIVLTQSPATLSLSPGERATLSCKSSQ |
| | | SLLDSGNQKNFLTWYQQKPGKAPKL |
| | | LIYWASTRESGVPSRFSGSGSGTDFT |
| | | FTISSLQPEDIATYYCQNDYSYPYTFG |
| | | QGTKVEIKRTVAAPSVFIFPPSDEQLK |
| | | SGTASVVCLLNNFYPREAKVQWKV |
| | | DNALQSGNSQESVTEQDSKDSTYSLS |
| | | STLTLSKADYEKHKVYACEVTHQGL |
| | | SSPVTKSFNRGEC |
| SEQ ID NO: 20 | DNA LC | GAGATCGTCCTGACTCAGTCACCC |
| | | GCTACCCTGAGCCTGAGCCCTGGC |
| | | GAGCGGGCTACACTGAGCTGTAAA |
| | | TCTAGTCAGTCACTGCTGGATAGCG |
| | | GTAATCAGAAGAACTTCCTGACCT |
| | | GGTATCAGCAGAAGCCCGGTAAAG |
| | | CCCCTAAGCTGCTGATCTACTGGGC |
| | | CTCTACTAGAGAATCAGGCGTGCC |
| | | CTCTAGGTTTAGCGGTAGCGGTAGT |
| | | GGCACCGACTTCACCTTCACTATCT |
| | | CTAGCCTGCAGCCCGAGGATATCG |
| | | CTACCTACTACTGTCAGAACGACTA |
| | | TAGCTACCCCTACACCTTCGGTCAA |
| | | GGCACTAAGGTCGAGATTAAGCGT |
| | | ACGGTGGCCGCTCCCAGCGTGTTC |
| | | ATCTTCCCCCCCAGCGACGAGCAG |
| | | CTGAAGAGCGGCACCGCCAGCGTG |
| | | GTGTGCCTGCTGAACAACTTCTACC |
| | | CCCGGGAGGCCAAGGTGCAGTGGA |
| | | AGGTGGACAACGCCCTGCAGAGCG |
| | | GCAACAGCCAGGAGAGCGTCACCG |
| | | AGCAGGACAGCAAGGACTCCACCT |
| | | ACAGCCTGAGCAGCACCCTGACCC |
| | | TGAGCAAGGCCGACTACGAGAAGC |
| | | ATAAGGTGTACGCCTGCGAGGTGA |
| | | CCCACCAGGGCCTGTCCAGCCCCG |
| | | TGACCAAGAGCTTCAACAGGGGCG |
| | | AGTGC |
| BAP049-Clone-E HC | | |
| SEQ ID NO: 21 (Kabat) | HCDR1 | TYWMH |
| SEQ ID NO: 22 (Kabat) | HCDR2 | NIYPGTGGSNFDEKFKN |
| SEQ ID NO: 23 (Kabat) | HCDR3 | WTTGTGAY |
| SEQ ID NO: 24 (Chothia) | HCDR1 | GYTFTTY |
| SEQ ID NO: 25 (Chothia) | HCDR2 | YPGTGG |
| SEQ ID NO: 26 (Chothia) | HCDR3 | WTTGTGAY |
| SEQ ID NO: 27 | VH | EVQLVQSGAEVKKPGESLRISCKGSG |
| | | YTFTTYWMHWVRQATGQGLEWMG |
| | | NIYPGTGGSNFDEKFKNRVTITADKS |
| | | TSTAYMELSSLRSEDTAVYYCTRWT |
| | | TGTGAYWGQGTTVTVSS |
| SEQ ID NO: 28 | DNA VH | GAGGTGCAGCTGGTGCAGTCAGGC |
| | | GCCGAAGTGAAGAAGCCCGGCGAG |
| | | TCACTGAGAATTAGCTGTAAAGGT |
| | | TCAGGCTACACCTTCACTACCTACT |
| | | GGATGCACTGGGTCCGCCAGGCTA |
| | | CCGGTCAAGGCCTCGAGTGGATGG |
| | | GTAATATCTACCCCGGCACCGGCG |
| | | GCTCTAACTTCGACGAGAAGTTTA |
| | | AGAATAGAGTGACTATCACCGCCG |
| | | ATAAGTCTACTAGCACCGCCTATAT |
| | | GGAACTGTCTAGCCTGAGATCAGA |
| | | GGACACCGCCGTCTACTACTGCACT |
| | | AGGTGGACTACCGGCACAGGCGCC |
| | | TACTGGGGTCAAGGCACTACCGTG |
| | | ACCGTGTCTAGC |
| SEQ ID NO: 29 | HC | EVQLVQSGAEVKKPGESLRISCKGSG |
| | | YTFTTYWMHWVRQATGQGLEWMG |
| | | NIYPGTGGSNFDEKFKNRVTITADKS |
| | | TSTAYMELSSLRSEDTAVYYCTRWT |
| | | TGTGAYWGQGTTVTVSSASTKGPSV |
| | | FPLAPCSRSTSESTAALGCLVKDYFP |
| | | EPVTVSWNSGALTSGVHTFPAVLQS |
| | | SGLYSLSSVVTVPSSSLGTKTYTCNV |
| | | DHKPSNTKVDKRVESKYGPPCPPCP |
| | | APEFLGGPSVFLFPPKPKDTLMISRTP |
| | | EVTCVVVDVSQEDPEVQFNWYVDG |
| | | VEVHNAKTKPREEQFNSTYRVVSVL |
| | | TVLHQDWLNGKEYKCKVSNKGLPS |
| | | SIEKTISKAKGQPREPQVYTLPPSQEE |
| | | MTKNQVSLTCLVKGFYPSDIAVEWE |
| | | SNGQPENNYKTTPPVLDSDGSFFLYS |
| | | RLTVDKSRWQEGNVFSCSVMHEAL |
| | | HNHYTQKSLSLSLG |
| SEQ ID NO: 30 | DNA HC | GAGGTGCAGCTGGTGCAGTCAGGC |
| | | GCCGAAGTGAAGAAGCCCGGCGAG |
| | | TCACTGAGAATTAGCTGTAAAGGT |
| | | TCAGGCTACACCTTCACTACCTACT |
| | | GGATGCACTGGGTCCGCCAGGCTA |
| | | CCGGTCAAGGCCTCGAGTGGATGG |
| | | GTAATATCTACCCCGGCACCGGCG |
| | | GCTCTAACTTCGACGAGAAGTTTA |
| | | AGAATAGAGTGACTATCACCGCCG |
| | | ATAAGTCTACTAGCACCGCCTATAT |
| | | GGAACTGTCTAGCCTGAGATCAGA |
| | | GGACACCGCCGTCTACTACTGCACT |
| | | AGGTGGACTACCGGCACAGGCGCC |
| | | TACTGGGGTCAAGGCACTACCGTG |
| | | ACCGTGTCTAGCGCTAGCACTAAG |
| | | GGCCCGTCCGTGTTCCCCCTGGCAC |
| | | CTTGTAGCCGGAGCACTAGCGAAT |
| | | CCACCGCTGCCCTCGGCTGCCTGGT |
| | | CAAGGATTACTTCCCGGAGCCCGT |
| | | GACCGTGTCCTGGAACAGCGGAGC |
| | | CCTGACCTCCGGAGTGCACACCTTC |
| | | CCCGCTGTGCTGCAGAGCTCCGGG |
| | | CTGTACTCGCTGTCGTCGGTGGTCA |
| | | CGGTGCCTTCATCTAGCCTGGGTAC |
| | | CAAGACCTACACTTGCAACGTGGA |
| | | CCACAAGCCTTCCAACACTAAGGT |
| | | GGACAAGCGCGTCGAATCGAAGTA |
| | | CGGCCCACCGTGCCCGCCTTGTCCC |
| | | GCGCCGGAGTTCCTCGGCGGTCCCT |
| | | CGGTCTTTCTGTTCCCACCGAAGCC |
| | | CAAGGACACTTTGATGATTTCCCGC |
| | | ACCCCTGAAGTGACATGCGTGGTC |
| | | GTGGACGTGTCACAGGAAGATCCG |
| | | GAGGTGCAGTTCAATTGGTACGTG |
| | | GATGGCGTCGAGGTGCACAACGCC |
| | | AAAACCAAGCCGAGGGAGGAGCA |
| | | GTTCAACTCCACTTACCGCGTCGTG |
| | | TCCGTGCTGACGGTGCTGCATCAG |
| | | GACTGGCTGAACGGGAAGGAGTAC |
| | | AAGTGCAAAGTGTCCAACAAGGGA |
| | | CTTCCTAGCTCAATCGAAAAGACC |
| | | ATCTCGAAAGCCAAGGGACAGCCC |
| | | CGGGAACCCCAAGTGTATACCCTG |
| | | CCACCGAGCCAGGAAGAAATGACT |
| | | AAGAACCAAGTCTCATTGACTTGC |
| | | CTTGTGAAGGGCTTCTACCCATCGG |
| | | ATATCGCCGTGGAATGGGAGTCCA |
| | | ACGGCCAGCCGGAAAACAACTACA |
| | | AGACCACCCCTCCGGTGCTGGACT |
| | | CAGACGGATCCTTCTTCCTCTACTC |
| | | GCGGCTGACCGTGGATAAGAGCAG |
| | | ATGGCAGGAGGGAAATGTGTTCAG |
| | | CTGTTCTGTGATGCATGAAGCCCTG |
| | | CACAACCACTACACTCAGAAGTCC |
| | | CTGTCCCTCTCCCTGGGA |
| BAP049-Clone-E LC | | |
| SEQ ID NO: 31 (Kabat) | LCDR1 | KSSQSLLDSGNQKNFLT |
| SEQ ID NO: 32 (Kabat) | LCDR2 | WASTRES |
| SEQ ID NO: 33 (Kabat) | LCDR3 | QNDYSYPYT |
| SEQ ID NO: 34 (Chothia) | LCDR1 | SQSLLDSGNQKNF |
| SEQ ID NO: 35 (Chothia) | LCDR2 | WAS |
| SEQ ID NO: 36 (Chothia) | LCDR3 | DYSYPY |
| SEQ ID NO: 37 | VL | EIVLTQSPATLSLSPGERATLSCKSSQ |
| | | SLLDSGNQKNFLTWYQQKPGQAPRL |
| | | LWWASTRESGVPSRFSGSGSGTDFT |
| | | FTISSLEAEDAATYYCQNDYSYPYTF |
| | | GQGTKVEIK |
| SEQ ID NO: 38 | DNA VL | GAGATCGTCCTGACTCAGTCACCC |
| | | GCTACCCTGAGCCTGAGCCCTGGC |
| | | GAGCGGGCTACACTGAGCTGTAAA |
| | | TCTAGTCAGTCACTGCTGGATAGCG |
| | | GTAATCAGAAGAACTTCCTGACCT |
| | | GGTATCAGCAGAAGCCCGGTCAAG |
| | | CCCCTAGACTGCTGATCTACTGGGC |
| | | CTCTACTAGAGAATCAGGCGTGCC |
| | | CTCTAGGTTTAGCGGTAGCGGTAGT |
| | | GGCACCGACTTCACCTTCACTATCT |
| | | CTAGCCTGGAAGCCGAGGACGCCG |
| | | CTACCTACTACTGTCAGAACGACTA |
| | | TAGCTACCCCTACACCTTCGGTCAA |
| | | GGCACTAAGGTCGAGATTAAG |
| SEQ ID NO: 39 | LC | EIVLTQSPATLSLSPGERATLSCKSSQ |
| | | SLLDSGNQKNFLTWYQQKPGQAPRL |
| | | LIYWASTRESGVPSRFSGSGSGTDFT |
| | | FTISSLEAEDAATYYCQNDYSYPYTF |
| | | GQGTKVEIKRTVAAPSVFIFPPSDEQL |
| | | KSGTASVVCLLNNFYPREAKVQWK |
| | | VDNALQSGNSQESVTEQDSKDSTYS |
| | | LSSTLTLSKADYEKHKVYACEVTHQ |
| | | GLSSPVTKSFNRGEC |
| SEQ ID NO: 40 | DNA LC | GAGATCGTCCTGACTCAGTCACCC |
| | | GCTACCCTGAGCCTGAGCCCTGGC |
| | | GAGCGGGCTACACTGAGCTGTAAA |
| | | TCTAGTCAGTCACTGCTGGATAGCG |
| | | GTAATCAGAAGAACTTCCTGACCT |
| | | GGTATCAGCAGAAGCCCGGTCAAG |
| | | CCCCTAGACTGCTGATCTACTGGGC |
| | | CTCTACTAGAGAATCAGGCGTGCC |
| | | CTCTAGGTTTAGCGGTAGCGGTAGT |
| | | GGCACCGACTTCACCTTCACTATCT |
| | | CTAGCCTGGAAGCCGAGGACGCCG |
| | | CTACCTACTACTGTCAGAACGACTA |
| | | TAGCTACCCCTACACCTTCGGTCAA |
| | | GGCACTAAGGTCGAGATTAAGCGT |
| | | ACGGTGGCCGCTCCCAGCGTGTTC |
| | | ATCTTCCCCCCCAGCGACGAGCAG |
| | | CTGAAGAGCGGCACCGCCAGCGTG |
| | | GTGTGCCTGCTGAACAACTTCTACC |
| | | CCCGGGAGGCCAAGGTGCAGTGGA |
| | | AGGTGGACAACGCCCTGCAGAGCG |
| | | GCAACAGCCAGGAGAGCGTCACCG |
| | | AGCAGGACAGCAAGGACTCCACCT |
| | | ACAGCCTGAGCAGCACCCTGACCC |
| | | TGAGCAAGGCCGACTACGAGAAGC |
| | | ATAAGGTGTACGCCTGCGAGGTGA |
| | | CCCACCAGGGCCTGTCCAGCCCCG |
| | | TGACCAAGAGCTTCAACAGGGGCG |
| | | AGTGC |

TABLE 2
Amino acid sequences of the heavy and light chain
leader sequences for humanized mAbs BAP049-Clone-B
and BAP049-Clone-E

| | | |
|---|---|---|
| BAP049- | HC | MAWVWTLPFLMAAAQSVQA (SEQ ID NO: 41) |
| Clone-B | LC | MSVLTQVLALLLLWLTGTRC (SEQ ID NO: 42) |
| BAP049- | HC | MAWVWTLPFLMAAAQSVQA (SEQ ID NO: 43) |
| Clone-E | LC | MSVLTQVLALLLLWLTGTRC (SEQ ID NO: 44) |

TABLE 3
Constant region amino acid sequences of human IgG heavy chains and
human kappa light chain

| | |
|---|---|
| HC | IgG4 (S228P) mutant constant region amino acid sequence (EU Numbering) |
| (SEQ | ASTKGPSVFP LAPCSRSTSE STAALGCLVK DYFPEPVTVS WNSGALTSGV |
| ID | HTFPAVLQSS GLYSLSSVVT VPSSSLGTKT YTCNVDHKPS NTKVDKRVES |
| NO: | KYGPPCPPCP APEFLGGPSV FLFPPKPKDT LMISRTPEVT CVVVDVSQED |
| 110) | PEVQFNWYVD GVEVHNAKTK PREEQFNSTY RVVSVLTVLH QDWLNGKEYK |
| | CKVSNKGLPS SIEKTISKAK GQPREPQVYT LPPSQEEMTK NQVSLTCLVK |
| | GFYPSDIAVE WESNGQPENN YKTTPPVLDS DGSFFLYSRL TVDKSRWQEG |
| | NVFSCSVMHE ALHNHYTQKS LSLSLGK |
| LC | Human kappa constant region amino acid sequence |
| (SEQ | RTVAAPSVFI FPPSDEQLKS GTASVVCLLN NFYPREAKVQ WKVDNALQSG |
| ID | NSQESVTEQD SKDSTYSLSS TLTLSKADYE KHKVYACEVT HQGLSSPVTK |
| NO: | SFNRGEC |
| 111) | |
| HC | IgG4 (S228P) mutant constant region amino acid sequence lacing C-terminal |
| (SEQ | lysine (K) (EU Numbering) |
| ID | ASTKGPSVFP LAPCSRSTSE STAALGCLVK DYFPEPVTVS WNSGALTSGV |
| NO: | HTFPAVLQSS GLYSLSSVVT VPSSSLGTKT YTCNVDHKPS NTKVDKRVES |
| 112) | KYGPPCPPCP APEFLGGPSV FLFPPKPKDT LMISRTPEVT CVVVDVSQED |
| | PEVQFNWYVD GVEVHNAKTK PREEQFNSTY RVVSVLTVLH QDWLNGKEYK |
| | CKVSNKGLPS SIEKTISKAK GQPREPQVYT LPPSQEEMTK NQVSLTCLVK |
| | GFYPSDIAVE WESNGQPENN YKTTPPVLDS DGSFFLYSRL TVDKSRWQEG |
| | NVFSCSVMHE ALHNHYTQKS LSLSLG |
| HC | IgG1 wild type |
| (SEQ | ASTKGPSVFP LAPSSKSTSG GTAALGCLVK DYFPEPVTVS WNSGALTSGV |
| ID | HTFPAVLQSS GLYSLSSVVT VPSSSLGTQT YICNVNHKPS NTKVDKRVEP |
| NO: | KSCDKTHTCP PCPAPELLGG PSVFLFPPKP KDTLMISRTP EVTCVVVDVS |
| 113) | HEDPEVKFNW YVDGVEVHNA KTKPREEQYN STYRVVSVLT VLHQDWLNGK |
| | EYKCKVSNKA LPAPIEKTIS KAKGQPREPQ VYTLPPSREE MTKNQVSLTC |
| | LVKGFYPSDI AVEWESNGQP ENNYKTTPPV LDSDGSFFLY SKLTVDKSRW |
| | QQGNVFSCSV MHEALHNHYT QKSLSLSPGK |
| HC | IgG1 (N297A) mutant constant region amino acid sequence (EU Numbering) |
| (SEQ | ASTKGPSVFP LAPSSKSTSG GTAALGCLVK DYFPEPVTVS WNSGALTSGV |
| ID | HTFPAVLQSS GLYSLSSVVT VPSSSLGTQT YICNVNHKPS NTKVDKRVEP |
| NO: | KSCDKTHTCP PCPAPELLGG PSVFLFPPKP KDTLMISRTP EVTCVVVDVS |
| 114) | HEDPEVKFNW YVDGVEVHNA KTKPREEQYA STYRVVSVLT VLHQDWLNGK |
| | EYKCKVSNKA LPAPIEKTIS KAKGQPREPQ VYTLPPSREE MTKNQVSLTC |
| | LVKGFYPSDI AVEWESNGQP ENNYKTTPPV LDSDGSFFLY SKLTVDKSRW |
| | QQGNVFSCSV MHEALHNHYT QKSLSLSPGK |
| HC | IgG1 (D265A, P329A) mutant constant region amino acid sequence (EU Numbering) |
| (SEQ | ASTKGPSVFP LAPSSKSTSG GTAALGCLVK DYFPEPVTVS WNSGALTSGV |
| ID | HTFPAVLQSS GLYSLSSVVT VPSSSLGTQT YICNVNHKPS NTKVDKRVEP |
| NO: | KSCDKTHTCP PCPAPELLGG PSVFLFPPKP KDTLMISRTP EVTCVVVAVS |
| 115) | HEDPEVKFNW YVDGVEVHNA KTKPREEQYN STYRVVSVLT VLHQDWLNGK |
| | EYKCKVSNKA LAAPIEKTIS KAKGQPREPQ VYTLPPSREE MTKNQVSLTC |
| | LVKGFYPSDI AVEWESNGQP ENNYKTTPPV LDSDGSFFLY SKLTVDKSRW |
| | QQGNVFSCSV MHEALHNHYT QKSLSLSPGK |
| HC | IgG1 (L234A, L235A) mutant constant region amino acid sequence (EU Numbering) |
| (SEQ | ASTKGPSVFP LAPSSKSTSG GTAALGCLVK DYFPEPVTVS WNSGALTSGV |
| ID | HTFPAVLQSS GLYSLSSVVT VPSSSLGTQT YICNVNHKPS NTKVDKRVEP |
| NO: | KSCDKTHTCP PCPAPEAAGG PSVFLFPPKP KDTLMISRTP EVTCVVVDVS |
| 116) | HEDPEVKFNW YVDGVEVHNA KTKPREEQYN STYRVVSVLT VLHQDWLNGK |
| | EYKCKVSNKA LPAPIEKTIS KAKGQPREPQ VYTLPPSREE MTKNQVSLTC |
| | LVKGFYPSDI AVEWESNGQP ENNYKTTPPV LDSDGSFFLY SKLTVDKSRW |
| | QQGNVFSCSV MHEALHNHYT QKSLSLSPGK |

TABLE 4
Amino acid sequences of the heavy and light chain framework regions
for humanized BAP049-Clone-B and BAP049-Clone-E

| | Amino Acid Sequence |
|---|---|
| VHFW1 | EVQLVQSGAEVKKPGESLRISCKGS (SEQ ID NO: 145) |
| (type a) | |
| VHFW1 | QVQLVQSGAEVKKPGASVKVSCKAS (SEQ ID NO: 49) |
| (type b) | |
| VHFW2 | WVRQATGQGLEWMG |
| (type a) | (SEQ ID NO: 51) |
| VHFW2 | W1RQSPSRGLEWLG |
| (type b) | (SEQ ID NO: 55) |
| VHFW2 | WVRQAPGQGLEWMG |
| (type c) | (SEQ ID NO: 58) |
| VHFW3 | RVTITADKSTSTAYMELSSLRSEDTAVYYCTR (SEQ ID NO: 60) |
| (type a) | |
| VHFW3 | RFTISRDNSKNTLYLQMNSLRAEDTAVYYCTR (SEQ ID NO: 64) |
| (type b) | |
| VHFW4 | WGQGTTVTVSS |
| | (SEQ ID NO: 67) |
| VLFW1 | EIVLTQSPDFQSVTPKEKVTITC (SEQ ID NO: 72) |
| (type a) | |
| VLFW1 | EIVLTQSPATLSLSPGERATLSC |
| (type b) | (SEQ ID NO: 75) |
| VLFW1 | DIVMTQTPLSLPVTPGEPASISC (SEQ ID NO: 79) |
| (type c) | |
| VLFW1 | DVVMTQSPLSLPVTLGQPASISC (SEQ ID NO: 81) |
| (type d) | |
| VLFW1 | DIQMTQSPSSLSASVGDRVTITC (SEQ ID NO: 83) |
| (type e) | |
| VLFW2 | WYQQKPGQAPRLLIY |
| (type a) | (SEQ ID NO: 85) |
| VLFW2 | WYQQKPGKAPKLLIY |
| (type b) | (SEQ ID NO: 89) |
| VLFW2 | WYLQKPGQSPQLLIY |
| (type c) | (SEQ ID NO: 92) |
| VLFW3 | GVPSRFSGSGSGTDFTFTISSLEAEDAATYYC (SEQ ID NO: 94) |
| (type a) | |
| VLFW3 | GIPPRFSGSGYGTDFTLTINNIESEDAAYYFC (SEQ ID NO: 98) |
| (type b) | |
| VLFW3 | GVPSRFSGSGSGTEFTLTISSLQPDDFATYYC (SEQ ID NO: 100) |
| (type c) | |
| VLFW3 | GVPSRFSGSGSGTDFTFTISSLQPEDIATYYC (SEQ ID NO: 103) |
| (type d) | |
| VLFW4 | FGQGTKVEIK (SEQ ID NO: 106) |

In one embodiment, the anti-PD-1 antibody molecule can include any of the following: a VH comprises a HCDR1 amino acid sequence of SEQ ID NO: 1, a HCDR2 amino acid sequence of SEQ ID NO: 2, and a HCDR3 amino acid sequence of SEQ ID NO: 3; and a VL comprising a LCDR1 amino acid sequence of SEQ ID NO: 11, a LCDR2 amino acid sequence of SEQ ID NO: 12, and a LCDR3 amino acid sequence of SEQ ID NO: 13; a VH comprising a HCDR1 amino acid sequence chosen from SEQ ID NO: 4; a HCDR2 amino acid sequence of SEQ ID NO:5; and a HCDR3 amino acid sequence of SEQ ID NO: 6; and a VL comprising a LCDR1 amino acid sequence of SEQ ID NO: 14, a LCDR2 amino acid sequence of SEQ ID NO: 15, and a LCDR3 amino acid sequence of SEQ ID NO: 16;

a VH comprising a HCDR1 amino acid sequence of SEQ ID NO: 21, a HCDR2 amino acid sequence of SEQ ID NO: 22, and a HCDR3 amino acid sequence of SEQ ID NO: 23; and a VL comprising a LCDR1 amino acid sequence of SEQ ID NO: 31, a LCDR2 amino acid sequence of SEQ ID NO: 32, and a LCDR3 amino acid sequence of SEQ ID NO: 33;

or

a VH comprising a HCDR1 amino acid sequence of SEQ ID NO: 24; a HCDR2 amino acid sequence of SEQ ID NO: 25; and a HCDR3 amino acid sequence of SEQ ID NO: 26; and a VL comprising a LCDR1 amino acid sequence of SEQ ID NO: 34, a LCDR2 amino. acid sequence of SEQ ID NO: 35, and a LCDR3 amino acid sequence of SEQ ID NO: 36.

In other embodiments, the aforesaid antibodies comprise a heavy chain variable domain comprising an amino acid sequence at least 85% identical to any of SEQ ID NOs: 7 or 27.

In other embodiments, the aforesaid antibody molecules comprise a light chain variable domain comprising an amino acid sequence at least 85% identical to any of SEQ ID NOs: 17 or 37.

In other embodiments, the aforesaid antibody molecules comprise a heavy chain variable domain comprising the amino acid sequence of SEQ ID NO: 7.

In other embodiments, the aforesaid antibody molecules comprise a heavy chain comprising the amino acid sequence of SEQ ID NO: 9.

In other embodiments, the aforesaid antibody molecules comprise a light chain variable domain comprising the amino acid sequence of SEQ ID NO: 17.

In other embodiments, the aforesaid antibody molecules comprise a light chain comprising the amino acid sequence of SEQ ID NO: 19.

In other embodiments, the aforesaid antibody molecules comprise a heavy chain variable domain comprising the amino acid sequence of SEQ ID NO: 27.

In other embodiments, the aforesaid antibody molecules comprise a heavy chain comprising the amino acid sequence of SEQ ID NO: 29.

In other embodiments, the aforesaid antibody molecules comprise a light chain variable domain comprising the amino acid sequence of SEQ ID NO: 37.

In other embodiments, the aforesaid antibody molecules comprise a light chain comprising the amino acid sequence of SEQ ID NO: 39.

In other embodiments, the aforesaid antibody molecules comprise a heavy chain variable domain comprising the amino acid sequence of SEQ ID NO: 7 and a light chain variable domain comprising the amino acid sequence of SEQ ID NO: 17.

In other embodiments, the aforesaid antibody molecules comprise a heavy chain comprising the amino acid sequence of SEQ ID NO: 9 and a light chain comprising the amino acid sequence of SEQ ID NO: 19.

In other embodiments, the aforesaid antibody molecules comprise a heavy chain variable domain comprising the amino acid sequence of SEQ ID NO:27 and a light chain variable domain comprising the amino acid sequence of SEQ ID NO: 37.

In other embodiments, the aforesaid antibody molecules comprise a heavy chain comprising the amino acid sequence of SEQ ID NO: 29 and a light chain comprising the amino acid sequence of SEQ ID NO: 39.

In other embodiments, the aforesaid antibody molecules are chosen from a Fab, F(ab′)2, Fv, or a single chain Fv fragment (scFv).

In other embodiments, the aforesaid antibody molecules comprise a heavy chain constant region selected from IgG1, IgG2, IgG3, and IgG4.

In other embodiments, the aforesaid antibody molecules comprise a light chain constant region chosen from the light chain constant regions of kappa or lambda.

In other embodiments, the aforesaid antibody molecules comprise a human IgG4 heavy chain constant region with a mutation at position 228 and a kappa light chain constant region.

In other embodiments, the aforesaid antibody molecules comprise a human IgG4 heavy chain constant region with a Serine to Proline mutation at position 228 or 214 and a kappa light chain constant region.

In other embodiments, the aforesaid antibody molecules comprise a human IgG1 heavy chain constant region with an Asparagine to Alanine mutation at position 297 and a kappa light chain constant region.

In other embodiments, the aforesaid antibody molecules comprise a human IgG1 heavy chain constant region with an Aspartate to Alanine mutation, and Proline to Alanine mutation of SEQ ID NO: 217 and a kappa light chain constant region.

In other embodiments, the aforesaid antibody molecules comprise a human IgG1 heavy chain constant region with a Leucine to Alanine mutation at position 234 and Leucine to Alanine mutation at position 235 and a kappa light chain constant region.

In other embodiments, the aforesaid antibody molecules are capable of binding to human PD-1 with a dissociation constant (K_{D}) of less than about 0.2 nM.

In some embodiments, the aforesaid antibody molecules bind to human PD-1 with a K_{D} of less than about 0.2 nM, 0.15 nM, 0.1 nM, 0.05 nM, or 0.02 nM, e.g., about 0.13 nM to 0.03 nM, e.g., about 0.077 nM to 0.088 nM, e.g., about 0.083 nM, e.g., as measured by a Biacore method.

In other embodiments, the aforesaid antibody molecules bind to cynomolgus PD-1 with a K_{D} of less than about 0.2 nM, 0.15 nM, 0.1 nM, 0.05 nM, or 0.02 nM, e.g., about 0.11 nM to 0.08 nM, e.g., about 0.093 nM, e.g., as measured by a Biacore method.

In certain embodiments, the aforesaid antibody molecules bind to both human PD-1 and cynomolgus PD-1 with similar K_{D}, e.g., in the nM range, e.g., as measured by a Biacore method. In some embodiments, the aforesaid antibody molecules bind to a human PD-1-Ig fusion protein with a K_{D} of less than about 0.1 nM, 0.075 nM, 0.05 nM, 0.025 nM, or 0.01 nM, e.g., about 0.04 nM, e.g., as measured by ELISA.

In some embodiments, the aforesaid antibody molecules bind to Jurkat cells that express human PD-1 (e.g., human PD-1-transfected Jurkat cells) with a K_{D} of less than about 0.1 nM, 0.075 nM, 0.05 nM, 0.025 nM, or 0.01 nM, e.g., about 0.06 nM, e.g., as measured by FACS analysis.

In some embodiments, the aforesaid antibody molecules bind to cynomolgus T cells with a K_{D} of less than about 1 nM, 0.75 nM, 0.5 nM, 0.25 nM, or 0.1 nM, e.g., about 0.4 nM, e.g., as measured by FACS analysis.

In some embodiments, the aforesaid antibody molecules bind to cells that express cynomolgus PD-1 (e.g., cells transfected with cynomolgus PD-1) with a K_{D} of less than about 1 nM, 0.75 nM, 0.5 nM, 0.25 nM, or 0.01 nM, e.g., about 0.6 nM, e.g., as measured by FACS analysis.

In certain embodiments, the aforesaid antibody molecules are not cross-reactive with mouse or rat PD-1. In other embodiments, the aforesaid antibodies are cross-reactive with rhesus PD-1. For example, the cross-reactivity can be measured by a Biacore method or a binding assay using cells that expresses PD-1 (e.g., human PD-1-expressing 300.19 cells). In other embodiments, the aforesaid antibody molecules bind an extracellular Ig-like domain of PD-1.

In other embodiments, the aforesaid antibody molecules are capable of reducing binding of PD-1 to PD-L1, PD-L2, or both, or a cell that expresses PD-L1, PD-L2, or both. In some embodiments, the aforesaid antibody molecules reduce (e.g., block) PD-L1 binding to a cell that expresses PD-1 (e.g., human PD-1-expressing 300.19 cells) with an IC50 of less than about 1.5 nM, 1 nM, 0.8 nM, 0.6 nM, 0.4 nM, 0.2 nM, or 0.1 nM, e.g., between about 0.79 nM and about 1.09 nM, e.g., about 0.94 nM, or about 0.78 nM or less, e.g., about 0.3 nM. In some embodiments, the aforesaid antibodies reduce (e.g., block) PD-L2 binding to a cell that expresses PD-1 (e.g., human PD-1-expressing 300.19 cells) with an IC50 of less than about 2 nM, 1.5 nM, 1 nM, 0.5 nM, or 0.2 nM, e.g., between about 1.05 nM and about 1.55 nM, or about 1.3 nM or less, e.g., about 0.9 nM.

In other embodiments, the aforesaid antibody molecules are capable of enhancing an antigen-specific T cell response.

In some embodiments, the aforesaid antibody molecules increase the expression of IL-2 from cells activated by Staphylococcal enterotoxin B (SEB) (e.g., at 25 μg/mL) by at least about 2, 3, 4, 5-fold, e.g., about 2 to 3-fold, e.g., about 2 to 2.6-fold, e.g., about 2.3-fold, compared to the expression of IL-2 when an isotype control (e.g., IgG4) is used, e.g., as measured in a SEB T cell activation assay or a human whole blood ex vivo assay.

In some embodiments, the aforesaid antibody molecules increase the expression of IFN-γ from T cells stimulated by anti-CD3 (e.g., at 0.1 μg/mL) by at least about 2, 3, 4, 5-fold, e.g., about 1.2 to 3.4-fold, e.g., about 2.3-fold, compared to the expression of IFN-γ when an isotype control (e.g., IgG4) is used, e.g., as measured in an IFN-γ activity assay.

In some embodiments, the aforesaid antibody molecules increase the expression of IFN-γ from T cells activated by SEB (e.g., at 3 pg/mL) by at least about 2, 3, 4, 5-fold, e.g., about 0.5 to 4.5-fold, e.g., about 2.5-fold, compared to the expression of IFN-γ when an isotype control (e.g., IgG4) is used, e.g., as measured in an IFN-γ activity assay.

In some embodiments, the aforesaid antibody molecules increase the expression of IFN-γ from T cells activated with an CMV peptide by at least about 2, 3, 4, 5-fold, e.g., about 2 to 3.6-fold, e.g., about 2.8-fold, compared to the expression of IFN-γ when an isotype control (e.g., IgG4) is used, e.g., as measured in an IFN-γ activity assay.

In some embodiments, the aforesaid antibody molecules increase the proliferation of CD8⁺ T cells activated with an CMV peptide by at least about 1, 2, 3, 4, 5-fold, e.g., about 1.5-fold, compared to the proliferation of CD8⁺ T cells when an isotype control (e.g., IgG4) is used, e.g., as measured by the percentage of CD8+ T cells that passed through at least n (e.g., n=2 or 4) cell divisions.

In certain embodiments, the aforesaid antibody molecules has a Cmax between about 100 μg/mL and about 500 μg/mL, between about 150 μg/mL and about 450 μg/mL, between about 250 μg/mL and about 350 μg/mL, or between about 200 μg/mL and about 400 μg/mL, e.g., about 292.5 μg/mL, e.g., as measured in monkey.

In certain embodiments, the aforesaid antibody molecules has a T_{1/2} between about 250 hours and about 650 hours, between about 300 hours and about 600 hours, between about 350 hours and about 550 hours, or between about 400 hours and about 500 hours, e.g., about 465.5 hours, e.g., as measured in monkey.

In some embodiments, the aforesaid antibody molecules bind to PD-1 with a Kd slower than 5×10⁻⁴, 1×10⁻⁴, 5×10⁻⁵, or 1×10⁻⁵ s⁻¹, e.g., about 2.13×10⁻⁴ s⁻¹, e.g., as measured by a Biacore method. In some embodiments, the aforesaid antibody molecules bind to PD-1 with a Ka faster than 1×10⁴, 5×10⁴, 1×10⁵, or 5×10⁵ M⁻¹s⁻¹, e.g., about 2.78×10⁵ M⁻¹s⁻¹, e.g., as measured by a Biacore method.

A preferred antibody molecule of the present invention is BAP049-Clone E.

Dosages and Dosing Regimens

The EGFR inhibitor, (R,E)-N-(7-chloro-1-(1-(4-(dimethylamino)but-2-enoyl)azepan-3-yl)-1H-benzo [d]imidazol-2-yl)-2-methylisonicotinamide (Compound A), or a pharmaceutically acceptable salt thereof, and the inhibitor of an immune checkpoint molecule, e.g., an inhibitor of PD-1 (e.g., an anti-PD-1 antibody molecule), may each be administered at a dose and/or on a time schedule, that in combination, achieves a desired anti-tumor activity.

The present invention provides the following dosing regimens.

Compound A may be administered at a dose between 5 mg and 100 mg, e.g., between 10 mg and 75 mg, between 15 mg and 50 mg, between 20 mg and 30 mg, between 10 mg and 40 mg, between 10 mg and 25 mg, or between 25 mg and 40 mg, e.g., at a dose of 5 mg, 10 mg, 15 mg, 20 mg, 25 mg, 30 mg, 35 mg, 40 mg, 45 mg, 50 mg, 60 mg, 70 mg, 80 mg, 90 mg, or 100 mg, e.g., twice a day, once a day, once every two days, once every three days, or once a week.

The anti-PD-1 antibody molecule, e.g. BAP049 Clone E, may be administered by injection (e.g., subcutaneously or intravenously) at a dose (e.g., a flat dose) of about 200 mg to 500 mg, e.g., about 250 mg to 450 mg, about 300 mg to 400 mg, about 250 mg to 350 mg, about 350 mg to 450 mg, or about 300 mg or about 400 mg. The dosing schedule (e.g., flat dosing schedule) can vary from e.g., once a week to once every 2, 3, 4, 5, or 6 weeks. For example, the anti-PD-1 antibody molecule is administered at a dose from about 300 mg to 400 mg once every three weeks or once every four weeks. In one embodiment, the anti-PD-1 antibody molecule is administered at a dose from about 300 mg once every four weeks. In one embodiment, the anti-PD-1 antibody molecule is administered at a dose from about 400 mg once every three weeks.

In one embodiment, the anti-PD-1 antibody molecule is administered at a dose from about 300 mg once every three weeks. In one preferred embodiment, the anti-PD-1 antibody molecule is administered at a dose from about 400 mg once every four weeks.

In one embodiment, the EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A) is administered at a dose between 10 mg and 50 mg (e.g., 25 mg), e.g., once a day. In some embodiments, the EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A), is administered orally. In one embodiment, the EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A), is administered at a dose between 10 mg and 50 mg (e.g., 25 mg), e.g., once a day, e.g., orally, and the PD-1 inhibitor (e.g., the anti-PD-1 antibody molecule, e.g. BAP049-Clone E) is administered at a dose between 300 mg and 500 mg (e.g., at a dose of 400 mg), e.g., once every 4 weeks, e.g., by intravenous infusion. In some embodiments, the combination is administered in one or more dosing cycles, e.g., one or more 28-day dosing cycles, e.g., one to six 28-day dosing cycles.

In certain embodiments, Compound A may not be administered on certain days of a given cycle. For example, in certain embodiments, the EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A), is administered on day 1 to day 5, or on day 1 to day 6, or on day 1 to day 7, or on day 1 to day 8, or on day 1 to day 9, preferably on day 1 to day 10 of any 28-day dosing cycle, e.g. the first 28-day dosing cycle. For example, the EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A), is administered at a dose of 25 mg, on day 1 to day 10 of any dosing cycle, e.g. the first dosing cycle. For example, the EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A), is administered at a dose of 50 mg, on day 1 to day 10 of any dosing cycle, e.g. the first dosing cycle.

In certain embodiments, the EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A), is not administered on day 11 to day 28 of a first dosing cycle, or in any subsequent dosing cycle(s).

Continuous therapy with a PD-1 inhibitor may prevent a durable anti-tumor immune response. Therefore, contemplated herein is a drug holiday or a treatment interruption period which is a period where neither Compound A nor the PD-1 inhibitor (e.g., the anti-PD-1 antibody molecule, e.g. BAP049-Clone E) is administered after a given dosing cycle.

For example, a drug holiday period is the period of days after the sequential administration of one of Compound A and the PD-1 inhibitor (e.g., the anti-PD-1 antibody molecule, e.g. BAP049-Clone E) and before the administration of the other of Compound A and the PD-1 inhibitor (e.g., the anti-PD-1 antibody molecule, e.g. BAP049-Clone E) where neither Compound A nor the PD-1 inhibitor (e.g., the anti-PD-1 antibody molecule, e.g. BAP049-Clone E) is administered. The drug holiday may, for example, be a period of days selected from: 1 day, 2 days, 3 days, 4 days, 5 days, 6 days, 7 days, 8 days, 9 days, 10 days, 11 days, 12 days, 13 days and 14 days.

The present invention therefore also provides a dosing regimen, wherein the treatment with the pharmaceutical combination is interrupted for a period or a drug holiday period, until evidence of disease progression emerges, wherein the pharmaceutical combination is administered upon evidence of disease progression. Disease progression may be measured e.g. by determining tumor response according to RECIST v 1.1. or irRC.

For example, Compound A, or Antibody B, or the combination therapy may be interrupted after 6 months and the patient followed for progression of disease. When there is a treatment interruption period or a drug holiday period, patients may continue safety and efficacy assessments until clinical or radiological evidence of disease progression emerges, at which time they may resume treatment.

Suitable diseases to be treated with the pharmaceutical combination of the present invention, and the dosing regimens described herein are colorectal cancer (CRC), a lung cancer (e.g., a non-small cell lung cancer (NSCLC)), or a breast cancer (e.g., a triple negative breast cancer (TNBC)). In some embodiments, the EGFR inhibitor, (R,E)-N-(7-chloro-1-(1-(4-(dimethylamino)but-2-enoyl)azepan-3-yl)-1H-benzo[d]imidazol-2-yl)-2-methylisonicotinamide (Compound A) is administered in combination with an inhibitor of PD-1 (e.g., an anti-PD-1 antibody molecule) to treat a colorectal cancer (CRC), a lung cancer (e.g., a non-small cell lung cancer (NSCLC)), or a breast cancer (e.g., a triple negative breast cancer (TNBC)).

Compositions

The present invention also relates to a pharmaceutical product or a commercial package comprising a combination product according to the invention described herein, in particular together with instructions for simultaneous, separate or sequential use (especially for being jointly active) thereof in the treatment of an EGFR tyrosine kinase activity mediated disease, especially a cancer.

The present invention embodiments also include pharmaceutically acceptable salts of the compounds useful according to the invention described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

A preferred salt of Compound A is the mesylate salt.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention, relates to a pharmaceutical combination, especially a pharmaceutical combination product, comprising the mentioned combination partners and at least one pharmaceutically acceptable carrier.

“Combination” refers to formulations of the separate partners with or without instructions for combined use or to combination products. The combination partners may thus be entirely separate pharmaceutical dosage forms or pharmaceutical compositions that are also sold independently of each other and where just instructions for their combined use are provided in the package equipment, e.g. leaflet or the like, or in other information e.g. provided to physicians and medical staff (e.g. oral communications, communications in writing or the like), for simultaneous or sequential use for being jointly active, especially as defined below.

“Combination product” includes a kit of parts for the combined administration where an anti-PD-1 antibody and Compound A, or a pharmaceutically acceptable salt thereof (and optionally yet a further combination partner (e.g. another drug as explained below, also referred to as “co-agent”) may be administered independently at the same time or separately within time intervals, especially where these time intervals allow that the combination partners show a cooperative (=joint), e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration and/or at the same time. The term “combination product” as used herein thus means a pharmaceutical product that results from the mixing or combining of more than one active ingredient and includes both fixed and non-fixed combinations of the active ingredients (which may also be combined).

The term “non-fixed combination” means that the active ingredients are both administered to a patient as separate entities either simultaneously, concurrently or sequentially with no specific time limits, wherein such administration provides therapeutically effective levels of the two com-pounds in the body of the patient. The latter also applies to cocktail therapy, e.g. the administration of three or more active ingredients.

The term “non-fixed combination” thus defines especially a “kit of parts” in the sense that the combination partners (i) an anti-PD-1 antibody and (ii) Compound A, or a pharmaceutically acceptable salt thereof (and if present further one or more co-agents) as defined herein can be dosed independently of each other or by use of different fixed combinations with distinguished amounts of the combination partners, i.e. simultaneously or at different time points, where the combination partners may also be used as entirely separate pharmaceutical dosage forms or pharmaceutical formulations that are also sold independently of each other and just instructions of the possibility of their combined use is or are provided in the package equipment, e.g. leaflet or the like, or in other information e.g. provided to physicians and 5 medical staff. The independent formulations or the parts of the kit of parts can then, e.g. be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (i) and (ii), thus being jointly active. The ratio of the total amounts of the combination partner (i) to the combination partner (ii) to be administered in the combined preparation can be varied, e.g. in order to cope with the needs of a patient sub-population to be treated or the needs of the single patient which different needs can be due to age, sex, body weight, etc. of the patients.

The combination partners (i) and (ii) in any embodiment are preferably formulated or used to be jointly (prophylactically or especially therapeutically) active. This means in particular that there is at least one beneficial effect, e.g. a mutual enhancing of the effect of the combination partners (i) and (ii), in particular a synergism, e.g. a more than additive effect, additional advantageous effects (e.g. a further therapeutic effect not found for any of the single compounds), less side effects, a combined therapeutic effect in a non-effective dosage of one or both of the combination partners (i) and (ii), and very preferably a clear synergism of the combination partners (i) and (ii). For example, the term “jointly (therapeutically) active” may mean that the compounds may be given separately or sequentially (in a chronically staggered manner, especially a sequence-specific manner) in such time intervals that they preferably, in the warm-blooded animal, especially human, to be treated, and still show a (preferably synergistic) interaction (joint therapeutic effect). A joint therapeutic effect can, inter alia, be determined by following the blood levels, showing that both compounds are present in the blood of the human to be treated at least during certain time intervals, but this is not to exclude the case where the compounds are jointly active although they are not present in blood simultaneously.

The present invention thus pertains to a combination product for simultaneous, separate or sequential use, such as a combined preparation or a pharmaceutical fixed combination, or a combination of such preparation and combination.

Moreover, the combination partners may be brought together into a combination therapy: (i) prior to release of the combination product to physicians (e.g. in the case of a kit comprising the compound of the invention and the other therapeutic agent); (ii) by the physician themselves (or under the guidance of a physician) shortly before administration; (iii) in the patient themselves, e.g. during sequential administration of the compound of the invention and the other therapeutic agent.

In certain embodiments, any of the above methods involve further administering one or more other (e.g. third) co-agents, especially a chemotherapeutic agent.

Also in this case, the combination partners forming a corresponding product according to the invention may be mixed to form a fixed pharmaceutical composition or they may be administered separately or pairwise (i.e. before, simultaneously with or after the other drug substance(s)).

A combination product according to the invention can besides or in addition be administered especially for cancer therapy in combination with chemotherapy, radiotherapy, immunotherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

In another aspect, the present invention provides compositions, e.g., pharmaceutically acceptable compositions, which include an antibody molecule described herein, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g. by injection or infusion).

The compositions of this invention may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical preferred compositions are in the form of injectable or infusible solutions. The preferred mode of administration is parenteral (e.g., intravenous, subcutaneous, intraperitoneal, intramuscular). In a preferred embodiment, the combination disclosed herein is administered by intravenous infusion or injection. In another preferred embodiment, the combination disclosed herein is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The combination disclosed herein can be administered by a variety of methods known in the art, although for many therapeutic applications, the preferred route/mode of administration is intravenous injection or infusion for the antibody and oral for Compound A For example, the antibody molecule can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the antibody molecules can be administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an antibody molecule is 0.1-30 mg/kg, more preferably 1-25 mg/kg. They can be delivered separately or simultaneously. In certain embodiments, the anti-PD-1 antibody molecule is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 3 to 10 mg/kg, 5 to 15 mg/kg, 10 to 20 mg/kg, 15 to 25 mg/kg, or about 3 mg/kg and Compound A, or a pharmaceutically acceptable salt thereof, is administered by injection (e.g., subcutaneously or intravenously) at a dose of about 1 to 40 mg/kg, e.g. 30 mg/kg. The dosing schedule can vary from e.g., once a week to once every 2, 3, or 4 weeks. The antibody molecules can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the infusion rate of about 110 to 130 mg/m² achieves a level of about 3 mg/kg. In other embodiments, the antibody molecules can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, the antibody is infused over a period of about 30 min.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the modified antibody or antibody fragment may vary according to factors such as the disease state, age, sex, and weight of the individual, and the ability of the antibody or antibody portion to elicit a desired response in the individual. A therapeutically effective amount is also one in which any toxic or detrimental effects of the modified antibody or antibody fragment is outweighed by the therapeutically beneficial effects. A “therapeutically effective dosage” of the disclosed combination preferably inhibits a measurable parameter, e.g., tumor growth rate by at least about 20%, more preferably by at least about 40%, even more preferably by at least about 60%, and still more preferably by at least about 80% relative to untreated subjects. The ability of the combination disclosed herein to inhibit a measurable parameter, e.g., cancer, can be evaluated in a clinical trial and evaluated by a skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the invention is a kit comprising an antibody molecule described herein. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

In one aspect, the invention relates to treatment of a subject in vivo using combination including an anti-PD-1 antibody molecule shown in Table 1 and Compound A, or a pharmaceutically acceptable salt thereof, such that growth of cancerous tumors as described herein are inhibited or reduced. The anti-PD-1 antibody and the Compound A, or a pharmaceutically acceptable salt thereof, combination can be used alone to inhibit the growth of cancerous tumors or can be used in combination with one or more of: a standard of care treatment (e.g., for cancers or infectious disorders), another antibody or antigen-binding fragment thereof, an immunomodulator (e.g., an activator of a costimulatory molecule or an inhibitor of an inhibitory molecule); a vaccine, e.g., a therapeutic cancer vaccinE other forms of cellular immunotherapy, as described below.

The combination described herein may be used for the treatment of lung cancer such as non-small cell lung cancer (NSCLC) or squamous lung cancer. The cancer may be locally advanced or metastatic NSCLC. In addition, the cancer may be resistant to treatment with erlotinib, gefitinib and/or icotinib. In addition, the cancer may be resistant to treatment with mereletinib and/or rociletinib.

Cancer subjects receiving the combination can be patients with lung cancer who have been previously treated with standard of care (e.g., erlotinib, gefitinib and icotinib) or patients who have not yet received any treatment. In one example, the combination described herein is used to treat patients having lung cancer who have been treated with standard of care but show disease progression.

The cancer to be treated may be cancer, e.g. NSCLC, with an EGFR mutation selected from the group consisting of L858R, ex19del and T790M, and combinations thereof. The predominant oncogenic EGFR mutations (L858R and ex19del) account for about 90% of EGFR NSCLC. A secondary “gatekeeper” T790M mutation may also develops in certain patients.

The combination described herein can be used for the treatment of a cancer which is resistant or refractory to immunotherapy such as anti-PD-1 or anti-PD-L1 therapy. Examples of these therapies include therapy with pembrolizumab, nivolumab, atezolizumab and MEDI4736.

In another embodiment, the combination of the invention can be administered alone or in combination with one or more other agents, and the combination can be administered in either order or simultaneously. In one example, the combination therapy disclosed herein can include a composition of the present invention co-formulated with, and/or co-administered with, one or more additional therapeutic agents, e.g., one or more anti-cancer agents, cytotoxic or cytostatic agents, hormone treatment, vaccines, and/or other immunotherapies. In other embodiments, the combination described herein can be administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

By “in combination with,” it is not intended to imply that the therapy or the therapeutic agents must be administered at the same time and/or formulated for delivery together, although these methods of delivery are within the scope described herein. The anti-PD-1 antibody and Compound A, or a pharmaceutically acceptable salt thereof can be administered concurrently with, prior to, or subsequent to, one or more other additional therapies or therapeutic agents. The anti-PD-1 antibody and Compound A, or a pharmaceutically acceptable salt thereof and the other agent or therapeutic protocol can be administered in any order. In general, each agent will be administered at a dose and/or on a time schedule determined for that agent. In will further be appreciated that the additional therapeutic agent utilized may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

In certain embodiments, the combination of the invention is administered in combination with one or more other inhibitors of PD-1, PD-L1 and/or PD-L2 known in the art. The antagonist may be an antibody, an antigen binding fragment thereof, an immunoadhesin, a fusion protein, or oligopeptide. In some embodiments, the other anti-PD-1 antibody is chosen from MDX-1106, Merck 3475 or CT-011. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 inhibitor is AMP-224. In some embodiments, the PD-L1 inhibitor is anti-PD-L1 antibody. In some embodiments, the anti-PD-L1 binding antagonist is chosen from YW243.55.S70, MPDL3280A, MEDI-4736, MSB-0010718C, or MDX-1105. MDX-1105, also known as BMS-936559, is an anti-PD-L1 antibody described in WO2007/005874. Antibody YW243.55.S70 (heavy and light chain variable region sequences shown in SEQ ID Nos. 20 and 21, respectively) is an anti-PD-L1 described in WO 2010/077634.

MDX-1106, also known as MDX-1106-04, ONO-4538 or BMS-936558, is an anti-PD-1 antibody described in WO2006/121168. Merck 3745, also known as MK-3475 or SCH-900475, is an anti-PD-1 antibody described in WO2009/114335. Pidilizumab (CT-011; Cure Tech) is a humanized IgG1k monoclonal antibody that binds to PD-1. Pidilizumab and other humanized anti-PD-1 monoclonal antibodies are disclosed in WO2009/101611. In other embodiments, the anti-PD-1 antibody is pembrolizumab. Pembrolizumab (Trade name Keytruda formerly lambrolizumab—also known as MK-3475) disclosed, e.g., in Hamid, O. et al. (2013) New England Journal of Medicine 369 (2): 134-44. AMP-224 (B7-DCIg; Amplimmune; e.g., disclosed in WO2010/027827 and WO2011/066342), is a PD-L2 Fc fusion soluble receptor that blocks the interaction between PD-1 and B7-H1. Other anti-PD-1 antibodies include AMP 514 (Amplimmune), among others, e.g., anti-PD-1 antibodies disclosed in U.S. Pat. No. 8,609,089, US 2010028330, and/or US 20120114649.

Exemplary other agents that can be combined with the combination of the invention can include standard of care chemotherapeutic agent including, but not limited to, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), vinorelbine (Navelbine®), Ibrutinib, idelalisib, and brentuximab vedotin.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids that can be used in combination with the anti-PD-1 antibody molecules, alone or in combination with another immunomodulator (e.g., an anti-LAG-3, anti-PD-L1 or anti-TIM-3 antibody molecule), include, but ate not limited to, vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary doses for the three (or more) agent regimens are as follows. The PD-1 antibody molecule can be administered, e.g., at a dose of about 1 to 40 mg/kg, e.g., 1 to 30 mg/kg, e.g., about 5 to 25 mg/kg, about 10 to 20 mg/kg, about 1 to 5 mg/kg, or about 3 mg/kg.

Biomarkers

The invention further includes selecting patients that may benefit most from treatment with the combination of the invention. Selection of patients can be achieved by determining for the presence of PD-1 or the presence of TAMS. While not wishing to be bound by theory, in some embodiments, a patient is more likely to respond to treatment with the combination of the invention if the patient has a cancer that highly expresses PD-L1, and/or the cancer is infiltrated by anti-tumor immune cells, e.g., TILs and/or has a high TAMS level, e.g., determined by looking for CD163 or CD163/CD8 as described below.

Selection of patients having PD-1

In one example, determining for the presence of PD-1 can be to determine the anti-tumor immune cells by assaying for cells positive for CD8, PD-L1, and/or IFN-γ; thus levels of CD8, PD-L1, and/or IFN-γ can serve as a readout for levels of TILs in the microenvironment. In certain embodiments, the cancer microenvironment is referred to as triple-positive for PD-L1/CD8/IFN-γ.

Accordingly, in certain aspects, this application provides methods of determining whether a tumor sample is positive for one or more of PD-L1, CD8, and IFN-γ, and if the tumor sample is positive for one or more, e.g., two, or all three, of the markers, then administering to the patient a therapeutically effective amount of an anti-PD-1 antibody molecule, optionally in combination with one or more other immunomodulators or anti-cancer agents.

In the following indications, a large fraction of patients are triple-positive for PD-L1/CD8/IFN-γ: TN breast cancer. Regardless of whether a large or small fraction of patients is triple-positive for these markers, screening the patients for these markers allows one to identify a fraction of patients that has an especially high likelihood of responding favorably to therapy with a PD-1 antibody (e.g., a blocking PD-1 antibody) in combination with Compound A and optionally one or more other immunomodulators (e.g., an anti-TIM-3 antibody molecule, an anti-LAG-3 antibody molecule, or an anti-PD-L1 antibody molecule) and/or anti-cancer agents.

In some embodiments, the cancer sample is classified as triple-positive for PD-L1/CD8/IFN-γ. This measurement can roughly be broken down into two thresholds: whether an individual cell is classified as positive, and whether the sample as a whole is classified as positive. First, one can measure, within an individual cell, the level of PD-L1, CD8, and/or IFN-γ. In some embodiments, a cell that is positive for one or more of these markers is a cell that has a higher level of the marker compared to a control cell or a reference value. For example, in some embodiments, a high level of PD-L1 in a given cell is a level higher than the level of PD-L1 in a corresponding non-cancerous tissue in the patient. As another example, in some embodiments, a high level of CD8 or IFN-γ in a given cell is a level of that protein typically seen in a TIL. Second, one can also measure the percentage of cells in the sample that are positive for PD-L1, CD8, and/or IFN-γ. (It is not necessary for a single cell to express all three markers.) In some embodiments, a triple positive sample is one that has a high percentage of cells, e.g., higher than a reference value or higher than a control sample, that are positive for these markers.

In other embodiments, one can measure the levels of PD-L1, CD8, and/or IFN-γ overall in the sample. In this case, a high level of CD8 or IFN-γ in the sample can be the level of that protein typically seen in a tumor infiltrated with TIL. Similarly, a high level of PD-L1 can be the level of that protein typically seen in a tumor sample, e.g., a tumor microenvironment.

The identification of subsets of patients that are triple-positive for PD-L1/CD8/IFN-γ reveals certain sub-populations of patients that are likely to be responsive to PD-1 antibody therapy. For instance, many IM-TN (immunomodulatory, triple negative) breast cancer patients are triple-positive for PD-L1/CD8/IFN-γ. IM-TN breast cancer is described in, e.g., Brian D. Lehmann et al., “Identification of human triple-negative breast cancer subtypes and preclinical models for selection of targeted therapies”, J Clin Invest. Jul. 1, 2011; 121(7): 2750-2767. Triple-negative breast cancers are those that do not express estrogen receptor (ER), progesterone receptor (PR) and Her2/neu. These cancers are difficult to treat because they are typically not responsive to agents that target ER, PR, and Her2/neu. Triple-negative breast cancers can be further subdivided into different classes, one of which is immunomodulatory. As described in Lehmann et al., IM-TN breast cancer is enriched for factors involved in immune cell processes, for example, one or more of immune cell signaling (e.g., TH1/TH2 pathway, NK cell pathway, B cell receptor signaling pathway, DC pathway, and T cell receptor signaling), cytokine signaling (e.g., cytokine pathway, IL-12 pathway, and IL-7 pathway), antigen processing and presentation, signaling through core immune signal transduction pathways (e.g., NFKB, TNF, and JAK/STAT signaling), genes involved in T-cell function, immune transcription, interferon (IFN) response and antigen processing. Accordingly, in some embodiments, the cancer treated is a cancer that is, or is determined to be, positive for one or more marker of IM-TN breast cancer, e.g., a factor that promotes one or more of immune cell signaling (e.g., TH1/TH2 pathway, NK cell pathway, B cell receptor signaling pathway, DC pathway, and T cell receptor signaling), cytokine signaling (e.g., cytokine pathway, IL-12 pathway, and IL-7 pathway), antigen processing and presentation, signaling through core immune signal transduction pathways (e.g., NFKB, TNF, and JAK/STAT signaling), genes involved in T-cell function, immune transcription, interferon (IFN) response and antigen processing.

Selection of patients having EGFR mutations

Patients with tumors harboring EGFR activating mutation (e.g., L858R and/or exi9del) and/or an acquired EGFR T790M mutation may particularly benefit from the combination of the present invention.

EGFR mutation status may be determined by tests available in the art, e.g. QIAGEN Therascreen® EGFR test and the Cobas® EGFR Mutation Test v2. The therascreen EGFR RGQ PCR Kit is an FDA-approved, qualitative real-time PCR assay for the detection of specific mutations in the EGFR oncogene. Evidence of EGFR mutation can be obtained from existing local data and testing of tumor samples. EGFR mutation status may be determined from any available tumor tissue.

Additional terms are defined below and throughout the application.

The term “Programmed Death 1” or “PD-1” include isoforms, mammalian, e.g., human PD-1, species homologs of human PD-1, and analogs comprising at least one common epitope with PD-1. The amino acid sequence of PD-1, e.g., human PD-1, is known in the art, e.g., Shinohara T et al. (1994) Genomics 23(3):704-6; Finger L R, et al. Gene (1997) 197(1-2):177-87.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The compositions and methods of the present invention encompass polypeptides and nucleic acids having the sequences specified, or sequences substantially identical or similar thereto, e.g., sequences at least 85%, 90%, 95% identical or higher to the sequence specified.

In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “antibody molecule” refers to a protein, e.g., an immunoglobulin chain or fragment thereof, comprising at least one immunoglobulin variable domain sequence. The term “antibody molecule” includes, for example, a monoclonal antibody (including a full length antibody which has an immunoglobulin Fc region). In an embodiment, an antibody molecule comprises a full length antibody, or a full length immunoglobulin chain. In an embodiment, an antibody molecule comprises an antigen binding or functional fragment of a full length antibody, or a full length immunoglobulin chain. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv); (viii) a single domain antibody. These antibody fragments are obtained using conventional techniques known to those with skill in the art, and the fragments are screened for utility in the same manner as are intact antibodies. The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

In an embodiment, an antibody molecule is a monospecific antibody molecule and binds a single epitope. E.g., a monospecific antibody molecule having a plurality of immunoglobulin variable domain sequences, each of which binds the same epitope.

In an embodiment an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule,

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope. In an embodiment the first epitope is located on PD-1 and the second epitope is located on a TIM-3, LAG-3, CEACAM (e.g., CEACAM-1 and/or CEACAM-5), PD-L1, or PD-L2.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

Generally, unless specifically indicated, the anti-PD-1 antibody molecules can include any combination of one or more Kabat CDRs and/or Chothia hypervariable loops, e.g., described in Table 1. In one embodiment, the following definitions are used for the anti-PD-1 antibody molecules described in Table 1: HCDR1 according to the combined CDR definitions of both Kabat and Chothia, and HCCDRs 2-3 and LCCDRs 1-3 according the CDR definition of Kabat. Under all definitions, each VH and VL typically includes three CDRs and four FRs, arranged from amino-terminus to carboxy-terminus in the following order: FR1, CDR1, FR2, CDR2, FR3, CDR3, FR4.

The term “antigen-binding site” refers to the part of an antibody molecule that comprises determinants that form an interface that binds to the PD-1 polypeptide, or an epitope thereof. With respect to proteins (or protein mimetics), the antigen-binding site typically includes one or more loops (of at least four amino acids or amino acid mimics) that form an interface that binds to the PD-1 polypeptide. Typically, the antigen-binding site of an antibody molecule includes at least one or two CDRs and/or hypervariable loops, or more typically at least three, four, five or six CDRs and/or hypervariable loops.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to PD-1. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

Effectiveness of the combination therapy disclosed herein, and emergence of disease progression, may be measured using RECIST criteria for tumor responses (Therasse P, Arbuck S, Eisenhauer E, et al (2000) New Guidelines to Evaluate the Response to Treatment in Solid Tumors, Journal of National Cancer Institute, Vol. 92; 205-16) and the revised RECIST 1.1 guidelines (Eisenhauer E, et al (2009). New response evaluation criteria in solid tumors: revised RECIST guideline (version 1.1). European Journal of Cancer; Vol. 45: 228-47.).

The primary analysis of the best overall response is based on the sequence of Investigator overall lesion responses. Based on the patients' best overall response during the study, the following rates are then calculated:

Overall response rate (ORR) is the proportion of patients with a best overall response of CR or PR. This is also referred to as ‘Objective response rate’ in some protocols or publications.

Disease control rate (DCR) is the proportion of patients with a best overall response of CR or PR or SD.

Another approach is to summarize the progression rate at a certain time point after baseline. In this case, the following definition is used:

Early progression rate (EPR) is the proportion of patients with progressive disease within 8 weeks of the start of treatment.

Tumor response assessment may also be determined locally according to immune-related Response Criteria (irRC) (Wolchok J D, Hoos A, O'Day S et al (2009) Guidelines for the Evaluation of Immune Therapy Activity in Solid Tumors: Immune-Related Response Criteria, Clin Cancer Res; 15:7412-20 and Nishino M, Giobbie-Hurder A, Gargano M, et al (2013) Developing a Common Language for Tumor Response to Immunotherapy: Immune-Related Response Criteria Using Unidimensional Measurements, Clin Cancer Res; 19:3936-3943.

Various aspects of the invention are described in further detail below. Additional definitions are set out throughout the specification.

### EXAMPLES

### Example 1: Characterization of Humanized Anti-PD-1 Antibody

Binding Affinity and Specificity

The Generation of humanized BAP049-Clone-B and BAP049-Clone E and characterization thereof is described in PCT application PCT/US2015/012754, which was published on 30 Jul. 2015, as WO/2015/112900.

Murine anti-PD-1 monoclonal antibody BAP049 was humanized. The sequences and test samples of sixteen humanized BAP049 clones with unique variable region sequences were obtained. These clones were further analyzed for their biological functions (e.g., antigen binding and ligand blocking), structural features, and transcient expression in CHO cells.

Binding Affinity and Specificity

The binding of an exemplary humanized anti-PD-1 antibody on human PD-1 protein was measured using Biacore method. The results are: Ka=2.78×10⁵ M⁻¹s⁻¹; Kd=2.13×10⁻⁴ s⁻¹ K_{D}=0.0827±0.005505 nM.

Humanization Technology and Process

Humanization of BAP049 was performed using a combinatorial library of human germline variable region frameworks (FWs). The technology entails transferring the murine CDRs in frame to a library of human variable regions (VRs) that had been constructed by randomly combining human germ line FW1, FW2 and FW3 sequences. Only one FW4 sequence was used, which is WGQGTTVTVSS (SEQ ID NO: 67) for the heavy chain (HC) (Kabat human HC subgroup I) and FGQGTKVEIK (SEQ ID NO: 106) for the light chain (LC) (Kabat human K subgroup I). The library of VR sequences was fused to human constant region (CR) sequences, human IgG4(S228P) of HC and human κ CR of LC, and the resulting library of whole IgG was expressed in CHO cells for screening. Screening was performed with tissue culture supernatants measuring binding avidity on antigen-expressing cells in a whole cell ELISA format or on FACS.

The humanization process was performed in a stepwise manner starting with the construction and expression of the appropriate chimeric mAb (murine VR, IgG4(S228P), human K), which can serve as a comparator for the screening of the humanized clones. The constant region amino acid sequences for human IgG4(S228P) heavy chain and human kappa light chain are shown in Table 3.

Humanization of the VR of LC and HC were performed in two independent steps. The library of humanized LC (huLC) was paired with the chimeric HC (murine VR, IgG4(S228P)) and the resulting “half-humanized” mAbs were screened for binding activity by ELISA. The huLC of clones with adequate binding activity (≥binding of chimeric mAb) were selected. Analogously, the library of humanized HC (huHC) was paired with the chimeric LC (murine VR, human K) and screened for binding activity by ELISA. The huHC of clones with appropriate binding activity (≥binding of chimeric mAb) were selected.

The variable regions of the selected huLC and huHC were then sequenced to identify the huLC and huHC with unique sequences (some clones from the initial selection process may share the same LC or HC). The unique huLC and huHC were then randomly combined to form a small library of humanized mAbs (humAbs), which was expressed in CHO cells and screened on antigen-expressing cells in an ELISA and FACS format. Clones with binding activities that were equal or better than the binding of the chimeric comparator mAb are the final product of the humanization process.

Construction of Chimeric Antibody

Three variants of the chimeric antibody were prepared that either had a Cys, Tyr or Ser residue at position 102 of the LC sequence. The three chimeric antibodies, i.e., BAP049-chi (Cys), BAP049-chi (Tyr), and BAP049-chi (Ser) (also known as BAP049-chi, BAP049-chi-Y, and BAP049-chi-S, respectively), were expressed in CHO cells and tested for their ability to compete with labeled murine antibody for binding to PD-1 expressing Jurkat cells. The three variants were indistinguishable in the competition experiment. The results show that the three chimeric mAbs (Cys, Tyr, Ser) compete equally well with the binding of the labeled murine mAb BAP049. The slight difference between the chimeric mAb curves and the murine mAb curve is probably due to the different methods used for determining mAb concentrations. The concentration of the murine mAb was determined by OD280 measurement, whereas the chimeric mAb concentrations in supernatants were determined with an ELISA using an IgG4 standard. The germline residue Tyr was selected for humanized antibodies.

Humanized Antibody Clones

The process of humanization yielded sixteen clones with binding affinities comparable to that of the chimeric antibody. In addition to binding data, for each clone, the VR sequences were provided along with a sample of the mAb. The samples had been prepared by transient transfections of CHO cells and were concentrated tissue culture supernatants. The antibody concentrations in the solutions had been determined by an IgG4-specific ELISA.

The sixteen unique clones are combinations of four unique HC sequences and nine unique LC sequences. For the HC FW regions, the HC sequences are combinations of one of two different VHFW1, one of three different VHFW2, and one of two different VHFW3 sequences. For the LC FW regions, the LC sequences are combinations of one of five different VLFW1, one of three different VLFW2, and one of four different VLFW3 sequences. The amino acid and nucleotide sequences of the heavy and light chain variable domains for the humanized BAP049 clones B and E are shown in Table 1. The amino acid and nucleotide sequences of the heavy and light chain CDRs of the humanized BAP049 clones are also shown in Table 1.

Analysis of Humanized Clones

Analysis of Binding Activity and Binding Specificity

The binding activity and specificity was measured in a competition binding assay using a constant concentration of Alexa 488-labeled murine mAb, serial dilutions of the test mAbs, and PD-1-expressing 300.19 cells. Incubations with the mAb mixtures having different concentration ratios of test mAb to labeled mAb was at 4° C. for 30 min. Bound labeled murine mAb was then quantified using a FACS machine. The experiment was performed twice. Within the accuracy of the experiment, all humanized clones show similar activity for competing with binding of labeled murine mAb. The activity is also comparable to the activity of the parent murine mAb and chimeric mAb. MAbs were ranked relative to each other. For example, it can be a weaker competitor if in both experiments the curve of a certain clone is to the right of the chimeric mAb curve or it can be a better competitor if the curve of a certain clone is to the left of the chimeric mAb curve.

Selection of Humanized Clones

Selected clones including clones B and E were further tested for their ability to block the binding of PD-L1 and PD-L2 to PD-1 and for enhancing T cell activity in vitro assays with human PBMC.

Blocking of Ligand Binding

Murine anti-PD-1 mAb blocks the binding of the natural ligands PD-L1 and PD-L2 to PD-1 expressed on cells at low concentrations. Whether the humanized clones had preserved the blocking capacity of the parent murine mAb was tested in comparative experiments with murine and chimeric antibodies.

The blocking capacity of the mAbs was evaluated in a competition binding assay using a constant concentration of PD-L1-huIgG1 Fc fusion protein or PD-L2-huIgG1 Fc fusion protein, serial dilutions of the mAbs to be tested, and PD-1-expressing 300.19 cells.

Incubation was at 4° C. for 30 min. Bound ligand fusion proteins were detected with PE-conjugated F(ab′)2 fragment of goat anti-human IgG which doesn't recognize IgG4 mAbs (Southern Biotech 2043-09), and flow cytometry. Within the accuracy of the experiments, the humanized clones, chimeric antibody and murine parent mAb demonstrated comparable blocking activity for both the PD-L1 and PD-L2 ligands.

Expression of Humanized Anti-PD-1 Antibody, BAP049

Five humanized clones were selected for evaluation of expression in Chinese Hamster Ovary (CHO) cells.

Single gene vectors (SGVs) were constructed using Lonza's GS Xceed vectors (IgG4proΔk for heavy chain and Kappa for light chain). The SGVs were amplified and transiently co-transfected into CHOK1SV GS-KO cells for expression at a volume of 2.8 L.

Expression cultures were harvested Day 6 post-transfection and clarified by centrifugation and sterile filtration. The clarified cell culture supernatant was purified using one-step Protein A chromatography. Product quality analysis in the form of SE-HPLC, SDS-PAGE, IEF, and LAL was carried out using purified material at a concentration of 1 mg/ml including an antibody as a control sample.

Vector Construction

The sequences of the light and heavy chain variable domain encoding regions were synthesised by GeneArt AG. Light chain variable domain encoding regions were sub-cloned into pXC-Kappa and heavy chain variable domain encoding regions into pXC-IgG4pro AK vectors respectively using the N-terminal restriction site Hind III and the C-terminal restriction sites BsiWI (light chain) and ApaI (heavy chain). Positive clones were screened by PCR amplification (primers 1053: GCTGACAGACTAACAGACTGTTCC (SEQ ID NO: 226) and 1072: CAAATGTGGTATGGCTGA (SEQ ID NO: 227)) and verified by restriction digest (using a double digest of EcoRI-HF and HindIII-HF) and nucleotide sequencing of the gene of interest.

DNA Amplification

A single bacterial colony was picked into 15 ml Luria Bertani (LB) medium (LB Broth, Sigma-Aldrich, L7275) containing 50 μg/ml ampicillin and incubated at 37° C. overnight with shaking at 220 rpm. The resulting starter culture was used to inoculate 1 L Luria Bertani (LB) medium containing 50 μg/ml ampicillin and incubated at 37° C. overnight with shaking at 220 rpm. Vector DNA was isolated using the QIAGEN Plasmid Plus Gigaprep system (QIAGEN, 12991). In all instances, DNA concentration was measured using a Nanodrop 1000 spectrophotometer (Thermo-Scientific) and adjusted to 1 mg/ml with EB buffer (10 mM Tris-Cl, pH 8.5). DNA quality for the single gene vectors was assessed by measuring the absorbance ratio A260/A280. This was found to be between 1.88 and 1.90.

Culture of CHOK1SV GS-KO Cells

CHOK1SV GS-KO cells were cultured in CD-CHO media (Invitrogen, 10743-029) supplemented with 6 mM glutamine (Invitrogen, 25030-123). Cells were incubated in a shaking incubator at 36.5° C., 5% CO₂, 85% humidity, 140 rpm. Cells were routinely sub-cultured every 3-4 days, seeding at 2×10⁵ cells/ml and were propagated in order to have sufficient cells available for transfection. Cells were discarded by passage 20.

Transient Transfections of CHOK1SV GS-KO Cells

Transient transfections were performed using CHOK1SV GS-KO cells which had been in culture a minimum two weeks. Cells were sub-cultured 24 h prior to transfection and cell viability was >99% at the time of transfection.

All transfections were carried out via electroporation using a Gene Pulse MXCell (Bio-Rad), a plate based system for electroporation. For each transfection, viable cells were resuspended in pre-warmed media to 2.86×10⁷ cells/ml. 80 μg DNA (1:1 ratio of heavy and light chain SGVs) and 700 μl cell suspension were aliquotted into each cuvette/well. Cells were electroporated at 300 V, 1300 μF. Transfected cells were transferred to pre-warmed media in Erlenmeyer flasks and the cuvette/wells rinsed twice with pre-warmed media which was also transferred to the flasks. Transfected cell cultures were incubated in a shaking incubator at 36.5° C., 5% CO₂, 85% humidity, 140 rpm for 6 days. Cell viability and viable cell concentrations were measured at the time of harvest using a Cedex HiRes automated cell counter (Roche).

Protein a Affinity Chromatography

Cell culture supernatant was harvested and clarified by centrifugation at 2000 rpm for 10 min, then filtered through a 0.22 μm PES membrane filter. Clarified supernatant was purified using a pre-packed 5 ml HiTrap MabSelect SuRE column (GE Healthcare, 11-0034-94) on an AKTA purifier (10 ml/min). The column was equilibrated with 50 mM sodium phosphate, 125 mM sodium chloride, pH 7.0 (equilibration buffer) for 5 column volumes (CVs). After sample loading, the column was washed with 2 CVs of equilibration buffer followed by 3 CVs of 50 mM sodium phosphate, 1 M sodium chloride pH 7.0 and a repeat wash of 2 CVs of equilibration buffer. The Product was then eluted with 10 mM sodium formate, pH 3.5 over 5 CVs. Protein containing, eluted fractions were immediately pH adjusted to pH 7.2 and filtered through a 0.2 μm filter.

A single protein-containing peak was observed during the elution phase. This peak was shown to contain the mAb, when analyzed by SE-HPLC and SDS-PAGE. Recovered protein yield is shown in Table 5. The clones expressed transiently in a range from 32.4 to 43.0 mg/L.

TABLE 5
Summary of yield, titre, monomer content and endotoxin levels

| | | | Monomer | Endotoxin levels |
|---|---|---|---|---|
| Product | Yield* (mg) | Titre* (mg/L) | Content (%) | (EU/mg) |
| Clone A | 107.5 | 38.38 | 93.94 | 0.04 |
| Clone B | 93.8 | 33.50 | 95.28 | 0.63 |
| Clone C | 90.7 | 32.38 | 97.83 | 0.04 |
| Clone D | 108.9 | 38.88 | 96.53 | 0.35 |
| Clone E | 120.4 | 43.00 | 97.73 | 0.14 |

*Post Protein A purification

SE-HPLC Analysis

Samples of Protein A purified antibodies were analyzed in duplicate by SE-HPLC on an Agilent 1200 series HPLC system, using a Zorbax GF-250 4 μm 9.4 mm ID×250 mm column (Agilent). Aliquots of sample at a concentration of 1 mg/ml were filtered through a 0.2 μm filter prior to injection. 80 μl aliquots were injected respectively and run at 1 ml/min for 15 minutes. Soluble aggregate levels were analysed using Chemstation (Agilent) software.

Chromatography profiles with retention time showing the percentage of the overall detected peak areas were obtained for the tested antibodies and a control IgG4 antibody. The products show a single protein peak at approximately 8.65 to 8.72 min comparable to the human IgG4 antibody control (about 8.64 min) and consistent with a monomeric antibody. Small amounts (up to about 4-5%) of higher molecular weight impurities, consistent with soluble aggregates, were detected at retention times between about 7.43 and 8.08 min.

SDS-PAGE Analysis

Reduced samples were prepared for analysis by mixing with NuPage 4×LDS sample buffer (Invitrogen, NP0007) and NuPage 10× sample reducing agent (Invitrogen, NP0009), and incubated at 70° C., 10 min. For non-reduced samples, the reducing agent and heat incubation were omitted. Samples were electrophoresed on 1.5 mm NuPage 4-12% Bis-Tris Novex pre-cast gels (Invitrogen, NP0335PK2) with NuPage MES SDS running buffer under denaturing conditions. 10 μl aliquots of SeeBlue Plus 2 pre-stained molecular weight standard (Invitrogen, LC5925) and a control IgG4 antibody at 1 mg/ml were included on the gel. 1 μl of each sample at 1 mg/ml were loaded onto the gel. Once electrophoresed, gels were stained with InstantBlue (TripleRed, ISB01L) for 30 min at room temperature. Images of the stained gels were analysed on a BioSpectrum Imaging System (UVP).

The analysis confirmed the presence of the antibody products and good levels of purity. Under non-reducing conditions, a predominant protein band close to 98 kDa was observed comparable with the control IgG4 antibody. The control IgG4 antibody and one tested clone display an additional fainter band corresponding to a heavy plus light chain half-antibody at approximately 70 kDa under non-reducing conditions. This is expected for the control antibody. Two bands were observed under reducing conditions consistent with the size of heavy (close to the position of the 49 kDa marker) and light chains (close to the position of the 28 kDa marker) and comparable with the bands found for the control IgG4 antibody.

Iso-electric Focussing (IEF) Analysis

Non-reduced samples of Protein A purified antibody were electrophoresed as described below.

5 μg of Protein A purified samples were electrophoresed on a 1.0 mm Novex pH 3-10 gradient gel (Invitrogen, EC66552BOX) using manufacturers recommended running conditions. A 10 μl aliquot of IEF pH 3-10 markers (Invitrogen, 39212-01) was included on the gel. Once electrophoresed, gels were fixed with 10% TCA solution for 30 min and then stained with InstantBlue (TripleRed, ISB01L) over night at room temperature. Images of the stained gels were analysed on a BioSpectrum Imaging System (UVP).

The tested clones show charge isoforms between pH 7.4 and 8.0 markers. The detected charge isoforms are slightly more basic than the theorectically calculated pIs for these antibodies which were predicted to be between 6.99 and 7.56. The general shift to more basic charge isoforms suggests the presence of post-translational modifications such as glycosylation on the molecules. Clone C and Clone E show comparable charge isoforms, which is also consistent with the theorectically calculated pI being the same for both (6.99).

The control IgG4 antibody behaved as expected.

TABLE 6
Charge isoforms as detected by Novex IEF analysis

| | pI of predominant | Acidic charge | Basic charge |
|---|---|---|---|
| Product | charge isoform* | isoforms* | isoforms* |
| Clone A | 7.6 | 2x; 7.5 to 7.55 | 7.7 |
| Clone B | 7.75 | 2x; 7.5 to 7.6 | 7.8 |
| Clone C | 7.5 | 2x; 7.4 to 7.5 | 7.55 |
| Clone D | 8.0 | 7.9 | 8.1 |
| Clone E | 7.5 | 2x; 7.4 to 7.5 | 7.55 |

*pI readings are estimated from the staining positions correlated against the IEF 3-10 marker.

Characterization of Humanized Anti-PD-1 Antibodies

Binding Affinity and Specificity

The binding of an exemplary humanized anti-PD-1 antibodies including Clone B and Clone E as shown in Table 1 on human PD-1 protein was measured using Biacore method. The results are: Ka=2.78×10⁵ M⁻¹s⁻¹; Kd=2.13×10⁻⁴ s⁻¹; K_{D}=0.0827±0.005505 nM.

The binding of the same humanized anti-PD-1 antibody on human PD-1-expressing 300.19 cells was measured using FACS analysis. The result shows that the anti-PD-1 antibody (human IgG4) binds with high affinity to human PD-1 compared to a human IgG4 isotype control.

The exemplary humanized anti-PD-1 antibody was found to exhibit high affinity to cynomolgus PD-1 protein and cynomolgus PD-1-expressing 300.19 cells. As measured by Biacore method, the anti-PD-1 antibody binds to cynomolgus PD-1 with a K_{D} of 0.093±0.015 nM. The binding affinity to cynomolgus PD-1 is comparable to its binding affinity to human PD-1.

Additional binding analyses show that the exemplary humanized anti-PD-1 antibody is not cross-reactive with mouse PD-1 or cross-reactive with parental cell line.

Blocking of Interactions Between PD-1 and its Ligands

The ability of the exemplary humanized anti-PD-1 antibody to block the interactions between PD-1 and both of its known ligands, PD-L1 and PD-L2 was examined. The results show that the anti-PD-1 antibody blocked the binding of PD-L1 and PD-L2 on human PD-1-expressing 300.19 cells compared to human IgG4 isotype control and no antibody control.

The anti-PD-1 antibody blocked PD-L1 binding on the 300.19 cells with an IC50 of 0.94±0.15 nM. The same antibody blocked PD-L2 binding on the 300.19 cells with an IC50 of 1.3±0.25 nM.

Cellular Activity

The ability of the exemplary humanized anti-PD-1 antibody to enhance the Staphylococcal enterotoxin B (SEB)-stimulated expression of IL-2 was tested in human whole blood ex vivo assay. Diluted human whole blood was incubated with the anti-PD-1 antibody in the presence or absence of SEB at 37° C. for 48 hours prior to IL-2 measurement.

The result shows that the anti-PD-1 antibody increased SEB-stimulated IL-2 expression by 2.28±0.32 fold compared to a human IgG4 isotype control (25 μg/ml SEB; n=5 donors).

### Example 2: EGF816 is a Potent Inhibitor of the TEC Family of Kinases

The Tec family kinases include ITK, BMX, TEC, RLK and BTK and are central in the propogation of T-cell receptor and chemokine receptor signaling. Compound A, a potent inhibitor of mutant EGFR, displays potent inhibition of Tec family kinases in vitro. As shown in Table 7, in the biochemical based assay, Compound A showed single digit nM potency on the three T-cell Tec family members: ITK, TEC and TXK. In the cellular assays, Compound A potently inhibited T-cell Tec family members with IC₅₀ values of 21, 107 and 140 nM in IL2-production, mouse CD4 T-cell and human CD4 T-cell proliferation, respectively. It was less potent on B-cell Tec family kinases, as demonstrated by up-shifted IC₅₀ values in mouse B-cell and TMD-8 (BTK-dependent) proliferation assays.

In vitro assay methods (assays described in Table 7):

The biochemical assays for ITK, TEC and TXK were carried out using Caliper Life Sciences' proprietary LabChip™ technology. This technology uses a microfluidic chip to measure the conversion of a fluorescent peptide substrate to a phosphorylated product. The product conversions were determined in the presence of various compound concentrations, and IC₅₀ values were calculated.

The cellular IL-2 Production assay was carried out using Jurkat cells. Upon CD3/CD28 stimulation overnight in the presence of various concentrations of compound, the IL-2 content in the conditioned media was measured by ELISA, and compound IC₅₀ was determined.

In the Mouse CD4 T cell assay, CD4+ T cells were purified from mouse spleens, and plated in the tissue culture plates coated with anti-CD3. Cells were incubated for 48h at 37° C. in the presence of various concentrations of compound. ³H-Thymidine was then added and cells were incubated for an additional 18h at 37° C. Cells were then harvested and read on a beta counter.

In the Human CD4 T cell assay, primary human CD4+ T cells isolated from a leukopak were cultured in the presence of anti-CD3/anti-CD28 beads to stimulate T cell proliferation. After 4 days, cell viability was measured using Cell Titer Glo.

In the Mouse B cell assay, B cells are purified from mouse splenocytes and plated in the tissue culture plates with supplement of anti-IgM and m-IL4. Cells were incubated at 37° C. in the presence of various concentrations of compound. After 3 days, cell viability was measured using Cell Titer Glo.

In the BTK-dependent TMD-8 cell proliferation assay, TMD-8 cells were incubated at 37° C. in the presence of various concentrations of compound. After 3 days, cell viability was measured using Cell Titer Glo.

TABLE 7
Compound A biochemical and cellular IC₅₀ on Tec family kinases

| Type | Assay | Compound A IC₅₀ (nM) |
|---|---|---|
| Cellular | IL2-Production* | 21 |
| | Mouse CD4 T cell | 107 |
| | Human CD4 T cell | 140 |
| | Mouse B cell | 295 |
| | BTK (TMD-8) | 224 |
| Biochemical | ITK | 1.3 |
| | TEC | 0.9 |
| | TXK | 2 |

*IL2-production assay encompasses the TEC-family kinases (ITK, TEC, and TXK)

### Example 3

T-cells play critical roles in immune regulation. T-cell Tec family kinases are important players in T-cell function, which in turn can modulate immune function. As Compound A showed potent inhibition of T-cell Tec family kinases, we further investigated its potential immune-modulatory effect in vivo. Compound A was tested in a T-cell dependent antibody response (TDAR) assay, a frequently used functional assessment of the immune system. Compound A was administered orally to rats for 5 weeks at a dose of 30 mg/kg/day. On study days 11 and 25 for the main study animals and days 28 and 42 for the recovery group, animals received 300 μg of KLH (Keyhole Limpet Hemocyanin) antigen. Samples for serology assessment of anti-KLH IgM and anti-KLH IgG antibodies (study days 19, 21, 23, 25 and 36 prior to dosing from the main study animals; recovery days 42 and 53 prior to KLH injection from the recovery animals) were collected. Immunomodulatory responses in Compound A-treated animals following KLH immunization were noted when values were compared to concurrent vehicle controls. As shown in Table 8, the decrease in anti-KLH IgM antibodies (primary response) peaked on study day 19-21 for all test groups in both male and female rats. The decreases were also observed in mean anti-KLH IgM values on study days 21, 23, 25 (primary response, time course) and day 36 (post boosting) for female rats. For anti-KLH IgG antibodies, decreases in mean concentration were apparent on study days 19, 21, 23 and 25 for both male and female rats. On study day 36, decreases in mean concentration were detected in female rats.

Recovery following withdrawal of Compound A treatment was noted. The Compound A-related decrease of anti-KLH antibody production in both male and female rats was reversible. This included both primary response-anti-KLH IgM, and isotype switch measured by secondary anti-KLH IgG production as indicated by values that were similar to concurrent controls at the recovery sampling time points (recovery day 42 and 53).

In summary, the in vivo effect of Compound A on primary IgM antibody formation and the isotype switch to IgG antibody was noted at 30 mg/kg. This effect was reversed following withdrawal of Compound A. Together, the in vitro biochemical/cellular data and in vivo TDAR results indicated that Compound A has potential immune-modulatory potential.

TABLE 8
Decreased anti-KLH IgM and IgG indicated by mean percent
differences compared to vehicle controls following
administration of Compound A at 30 mg/kg/day

| | |
|---|---|
| Males | Female |

| Study Days | IgM | IgG | IgM | IgG |
|---|---|---|---|---|
| 19 | −67 | −78 | −88 | −96 |
| 21 | — | −84 | −75 | −98 |
| 23 | — | −80 | −71 | −97 |
| 25 | — | −83 | −63 | −96 |
| 36 | — | — | −67 | −69 |
| 42* | — | — | — | — |
| 53* | — | — | — | — |

— indicates results were not considered meaningfully different from control results.
Mean % difference = (mean dose group value − mean control value)/mean control value) × 100%
*indicates recovery days

### Example 4: In Vivo Pharmacology for Combination of Anti-PD-L1 Antibody and Compound A

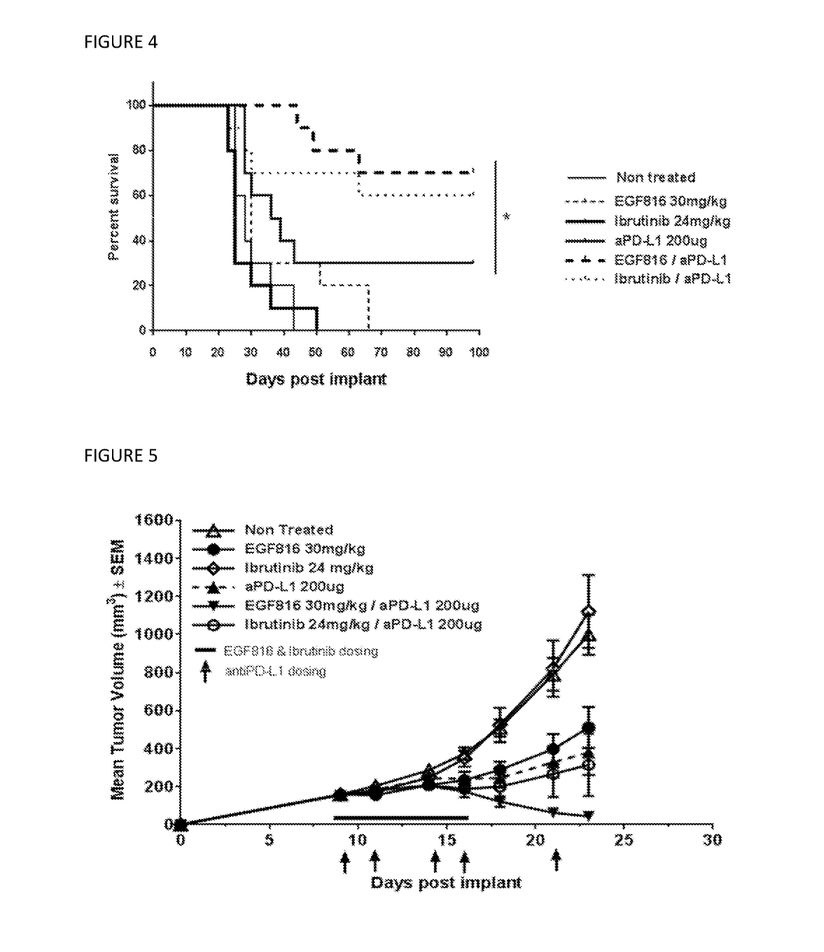

Compound A was combined in vivo with an exemplary anti-PD-L1 antibody molecule in an A20 lymphoma model. As shown in FIG. 4, the combination of anti-PD-L1 antibody and Compound A, or anti-PD-L1 antibody and ibrutinib, were more effective than any single agent. Compound A and ibrutinib were dosed for only ten days, and a total of 5 doses of anti-PD-L1-antibody were given. Even though Compound A and ibrutinib were only dosed transiently, the effects of Compound A plus anti-PD-L1 antibody and ibrutinib plus anti-PD-L1 antibody on survival extended beyond 60 days. As shown in FIG. 5, the combination of anti-PD-L1 antibody and Compound A also resulted in tumor regression in mice bearing A20 lymphoma allografts. In FIG. 5, the bar indicates treatment period for EGF816 and ibrutinib and arrows indicate when anti-PD-L1-antibody was administered.

The combination of COMPOUND A with anti-PD-L1 antibody was also well tolerated, and positive body weight change observed in animals treated at all doses during the course of treatment.

### Example 5: Pharmacokinetics Analysis of Flat Dosing Schedules

Based on pharmacokinetic (PK) modeling, utilizing flat dose is expected provide the exposure to patients at the appropriate Cmin concentrations. Over 99.5% of patients will be above EC50 and over 93% of patients will be above EC90. Predicted steady state mean Cmin for the exemplary anti-PD-1 antibody molecule (BAP049-Clone E) utilizing either 300 mg once every three weeks (Q3W) or 400 mg once every four weeks (Q4W) is expected to be above 20ug/mL (with highest weight, 150 kg) on average.

TABLE 9
Exemplary PK parameters based on flat dosing schedules

| | |
|---|---|
| Number of patients in PK dataset | 46 |
| CL (mL/h) | 10.9 [8.9, 13.2]; IIV: 62% |
| Exponent of Weight on CL | 0.54 [0.021, 1.06] |
| Volume of distribution at SS (L) | 7.2 [6.5, 7.9]; IIV: 22% |
| Half-Life (days) | 20 [17, 23]; IIV: 64% |
| Predicted Cmin (ug/mL) for 80 kg patient | 31 [22, 42] (400 mg q4w) |
| | 35 [26, 47] (300 mg q3w) |

The expected mean steady state Cmin concentrations for the exemplary anti-PD-1 antibody molecule observed with either doses/regimens (300 mg q3w or 400 mg q4w) will be at least 77 fold higher than the EC50 (0.42ug/mL) and about 8.6 fold higher than the EC90. The ex vivo potency is based on IL-2 change in SEB ex-vivo assay.

Less than 10% of patients are expected to achieve Cmin concentrations below 3.6ug/mL for either 300 mg Q3W or 400 mg Q4W. Less than 0.5% of patients are expected to achieve Cmin concentrations below 0.4 μg/mL for either 300 mg Q3W or 400 mg Q4W.

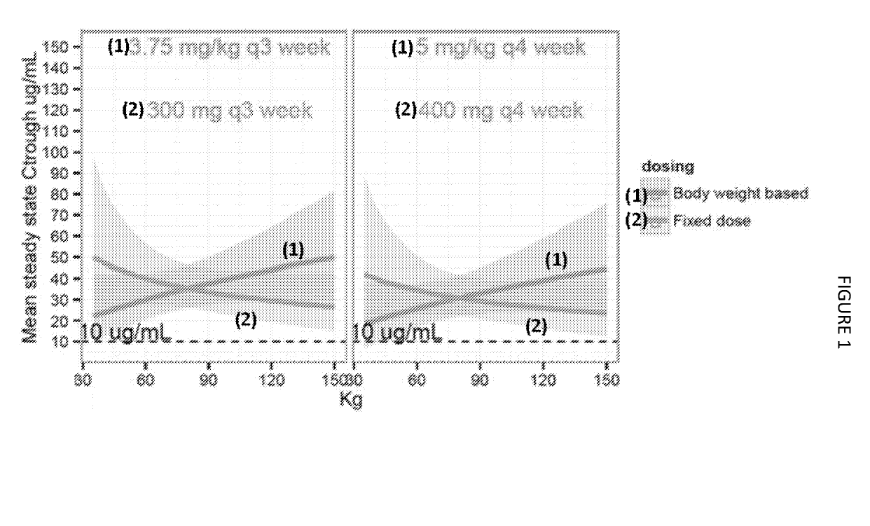

Predicted Ctrough (Cmin) concentrations across the different weights for patients while receiving the same dose of the exemplary anti-PD-1 antibody molecule are shown in FIG. 1. Body weight based dosing is compared to fixed dose (3.75 mg/kg Q3W vs. 300 mg Q3W and 5 mg/kg Q4W vs. 400 mg Q4W). FIG. 1 supports flat dosing of the exemplary anti-PD-1 antibody molecule.

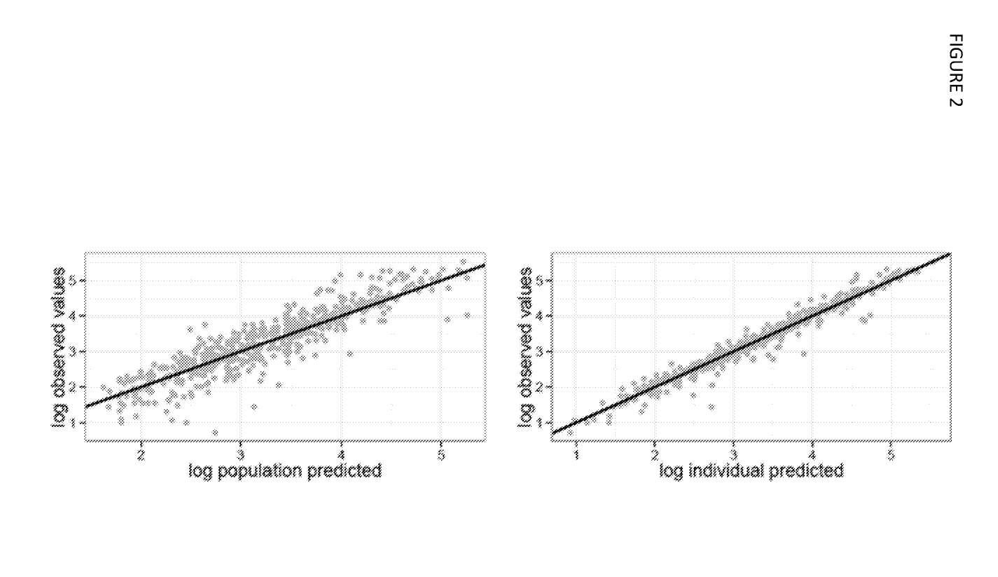

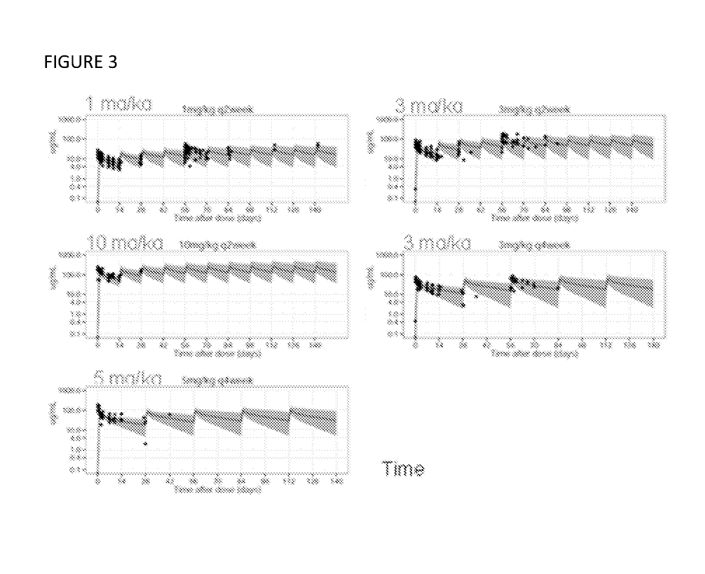

The PK model further is validated. As shown in FIG. 2, the observed versus model predicted concentrations lie on the line of unity. FIG. 3 shows that the model captures accumulation, time course, and within subject variability.

A recommended dose for the antibody molecule may therefore be selected as 400 mg Q4W. An alternative dosing regimen of 300 mg Q3W is expected to achieve similar exposure to 400 mg Q4W, and may be utilized in combination regimens where a Q3W schedule in a given dosing cycle is more convenient.

### Example 6: A Phase Ib, Open-Label, Multi-Center Study of Compound A Combined with an Exemplary Antibody Molecule (Antibody B) in Adult Patients with Colorectal Cancer (CRC), Non-Small-Cell Lung Cancer (NSCLC) or Triple-Negative Breast Cancer (TNBC)

For this study, the investigational drugs are Compound A and Antibody Molecule B, an anti-PD-1 receptor recombinant humanized monoclonal antibody.

The exemplary antibody molecule, Antibody B, (BAP049-Clone-E) tested in this study is a humanized anti-programmed death-1 (PD-1) IgG4 monoclonal antibody (mAb) that blocks binding of programmed cell death ligand-1 (PD-L1) and programmed cell death ligand-2 (PD-L2) to PD-1. It binds to PD-1 with high affinity and inhibits its biological activity. The amino acid sequences of this antibody molecule are described in Table 1 herein.

The study is comprised of a dose escalation part followed by a dose expansion part. The study treatment is administered in 28-day dosing cycles.

Study Periods

Patients enrolled in escalation part and expansion part participate in the following study periods:
Screening period
Treatment period 1—which may consist of up to 6 cycles
Treatment interruption period
Treatment period 2
Safety follow up period
Disease progression follow-up

The dosing cycle used throughout this study is a 28-day dosing cycle.

Screening Period

The screening period begins once the patient has signed the study informed consent. Patients are evaluated to ensure that they meet all the inclusion and none of the exclusion criteria.

Treatment Period 1

During treatment period 1, study treatment is administered for up to six cycles unless the patient experiences unacceptable toxicity, has clinical evidence of disease progression and/or treatment is discontinued at the discretion of the investigator or the patient. Patients who have radiological evidence of disease progression but have evidence of clinical benefit may continue study treatment to complete six cycles.

Treatment Interruption Period

Once a patient completes treatment period 1, study treatment is interrupted and the patient enters the study treatment-interruption period. Patients continue study visits for safety assessments (monthly) and tumor assessments (every 2 months).

Once a patient has clinical or radiological evidence of disease progression, they may resume treatment.

Treatment Period 2

Patients may resume study treatment at the same dose and schedule he/she was receiving prior to interrupting therapy. For patients receiving Antibody B+Compound A, study treatment in treatment period 2 is given as in treatment period 1 (in cycle 1 only). All patients have a tumor assessment, e.g. using RECIST v 1.1 or irRC criteria, prior to resuming study treatment.

This tumor assessment is used as treatment period 2 baseline scan.

Following the completion of two cycles of study treatment, if a patient has not experienced any>grade 2 study treatment-related toxicities, he/she may continue on study under a reduced schedule of assessments per the institution's standard of care or every three months, whichever is more frequent. Patients who have radiological disease progression during treatment period 2 and have evidence of clinical benefit may continue study treatment.

End of Treatment (EOT) Visit

An EOT visit occurs within 14 days of the decision to permanently discontinue study treatment regardless of whether the patient is in treatment period 1, treatment interruption period or treatment period 2. All participating patients must complete the EOT visit.

Dose and Treatment Schedule

Antibody B, as a lyophilisate in vial (LYVI) for i.v. infusion, is given at dose of 400 mg as a fixed dose, once every four weeks. Antibody B is given as a 30 minute i.v. infusion, or up to two hours if clinically indicated. Antibody B dose may be delayed by up to seven days.

Compound A can be administered before or after the Antibody B infusion.

Compound A is initially given at or below a low dose with evidence of pharmacologic activity established previously by other clinical studies. For example, the starting dose of Compound A may be 25 mg given daily, from Day 1 to Day 10 only in the first cycle, and then stopped. If the dose combination is determined to be safe, the dose of Compound A is tested in additional patients to confirm the safety and tolerability at that dose level, or escalated. For example, the starting dose of Compound A may be escalated to 50 mg given daily, from Day 1 to Day 10 in the first cycle, and then stopped. The dose escalation is guided by a Bayesian Logistic Regression Model (BLRM) based on any Dose Limiting Toxicities (DLTs) observed in the first two cycles of therapy. The BLRM is a well-established method to estimate the maximum tolerated dose (MTD)/recommended dose for expansion (RDE) in cancer patients. The adaptive BLRM is guided by the Escalation With Overdose Control (EWOC) principle to control the risk of DLT in future patients on the study. The use of Bayesian response adaptive models for small datasets has been accepted by EMA (Guideline on clinical trials in small populations Feb. 1 2007) and endorsed by numerous publications and its development and appropriate use is one aspect of the FDA's Critical Path Initiative.

The MTD is defined as the highest combination of drug doses not expected to cause DLT in 33% or more of the treated patients in 56 days following the first treatment of the combination.

Dose Expansion

Once the MTD/RDE is determined for the combination, the expansion part of the study is initiated to further assess the safety, tolerability and preliminary efficacy of the combination.

As a result, the dose of Compound A is expected to be identified without testing a large number of dose levels or schedules. To assess the pharmacodynamic activity of the combination, all patients undergo a tumor biopsy at baseline and again after approximately two cycles of therapy. In each target disease indication (CRC, NSCLC and TNBC) the extent of the change in tumor infiltration by immune cells including lymphocytes and myeloid cells may contribute to a decision on any potential benefit for a given combination.

Inclusion Criteria for Patients Eligible for Inclusion in this Study
1. Age≥18 years.
2. Patients with advanced/metastatic cancer, with measurable disease as determined by RECIST version 1.1, who have progressed despite standard therapy or are intolerant to standard therapy, or for whom no standard therapy exists. Patients must fit into one of the following groups:
   CRC (not mismatch repair deficient by local assay including PCR and/or IHC)
   NSCLC (adenocarcinoma)
   TNBC
3. Eastern Cooperative Oncology Group (ECOG) Performance Status≤2
4. Patient must have a site of disease amenable to biopsy, and be a candidate for tumor biopsy according to the treating institution's guidelines. Patient must be willing to undergo a new tumor biopsy at baseline, and again during therapy on this study.
5. Prior therapy with PD-1/PDL-1 inhibitors is allowed provided any toxicity attributed to prior PD-1- or PD-L1-directed therapy did not lead to discontinuation of therapy.

Principal Exclusion Criteria

1. Presence of symptomatic central nervous system (CNS) metastases, or CNS metastases that require local CNS-directed therapy (such as radiotherapy or surgery), or increasing doses of corticosteroids within the prior 2 weeks.

2. History of severe hypersensitivity reactions to other mAbs.

3. Patient having out of range laboratory values defined as:
Creatinine clearance (calculated using Cockcroft-Gault formula, or measured)<40 mL/min
Total bilirubin>1.5×ULN, except for patients with Gilbert's syndrome who are excluded if total bilirubin>3.0×ULN or direct bilirubin>1.5×ULN
Alanine aminotransferase (ALT)>3×ULN, except for patients that have tumor involvement of the liver, who are excluded if ALT>5×ULN
Aspartate aminotransferase (AST)>3×ULN, except for patients that have tumor involvement of the liver, who are excluded if AST>5×ULN
Absolute neutrophil count<1.0×10⁹/L without growth factor support
Platelet count<75×10⁹/L without growth factor or transfusion support
Hemoglobin (Hgb)<9 g/dL without growth factor or transfusion support
Potassium, magnesium, calcium or phosphate abnormality>CTCAE grade 1 despite appropriate replacement therapy appropriate replacement therapy

4. Impaired cardiac function or clinically significant cardiac disease, including any of the following:
Clinically significant and/or uncontrolled heart disease such as congestive heart failure requiring treatment (NYHA grade>2), uncontrolled hypertension or clinically significant arrhythmia
QTcF>470 msec for females or >450 msec for males on screening ECG or congenital long QT syndrome
Acute myocardial infarction or unstable angina pectoris<3 months prior to study entry

5. Patients with active, known or suspected autoimmune disease. Patients with vitiligo, type I diabetes mellitus, residual hypothyroidism due to autoimmune condition only requiring hormone replacement, psoriasis not requiring systemic treatment, or conditions not expected to recur in the absence of an external trigger are permitted to enroll.

6. Human Immunodeficiency Virus (HIV) infection at screening.

7. Escalation part: Active Hepatitis B (HBV) virus or Hepatitis C (HCV) virus infection at screening.

Expansion part: Patients with active HBV or HCV are excluded, excepting those patients undergoing treatment for HBV or HCV.

8. Malignant disease, other than that being treated in this study. Exceptions to this exclusion include the following: malignancies that were treated curatively and have not recurred within 2 years prior to study treatment; completely resected basal cell and squamous cell skin cancers; any malignancy considered to be indolent and that has never required therapy; and completely resected carcinoma in situ of any type.

9. Systemic anti-cancer therapy within 2 weeks of the first dose of study treatment. For cytotoxic agents that have major delayed toxicity, e.g. mitomycin C and nitrosoureas, 6 weeks is indicated as washout period. For patients receiving anticancer immunotherapies such as CTLA-4 antagonists, 6 weeks is indicated as the washout period.

10. Active infection requiring systemic antibiotic therapy.

11. Patients requiring chronic treatment with systemic steroid therapy, other than replacement dose steroids in the setting of adrenal insufficiency. Topical, inhaled, nasal and ophthalmic steroids are allowed.

12. Patients receiving systemic treatment with any immunosuppressive medication (other than steroids as described above).

13. Use of any live vaccines against infectious diseases (e.g. influenza, varicella, pneumococcus) within 4 weeks of initiation of study treatment.

The use of live vaccines is not allowed through the entire duration of the study.

14. Major surgery within 2 weeks of the first dose of study treatment (mediastinoscopy, insertion of a central venous access device, and insertion of a feeding tube are not considered major surgery).

15. Radiotherapy within 2 weeks of the first dose of study drug, except for palliative radiotherapy to a limited field, such as for the treatment of bone pain or a focally painful tumor mass. To allow for assessment of response to treatment, patients must have remaining measurable disease that has not been irradiated.

16. Participation in an interventional, investigational study within 2 weeks of the first dose of study treatment.

17. Presence of ≥CTCAE grade 2 toxicity (except alopecia, peripheral neuropathy and ototoxicity, which are excluded if≥CTCAE grade 3) due to prior cancer therapy.

18. Use of hematopoietic colony-stimulating growth factors (e.g. G-CSF, GMCSF, M-CSF)≤2 weeks prior start of study drug. An erythroid stimulating agent is allowed as long as it was initiated at least 2 weeks prior to the first dose of study treatment.

19. Any medical condition that would, in the investigator's judgment, prevent the patient's participation in the clinical study due to safety concerns, compliance with clinical study procedures or interpretation of study results.

20. Pregnant or lactating women, where pregnancy is defined as the state of a female after conception and until the termination of gestation, confirmed by a positive hCG laboratory test. In rare cases of an endocrine-secreting tumor, hCG levels may be above normal limits but with no pregnancy in the patient. In these cases, there should be a repeat serum hCG test (with a non-rising result) and a vaginal/pelvic ultrasound to rule out pregnancy. Upon confirmation of results and discussion with the Medical representative, these patients may enter the study.

21. Women of child-bearing potential, defined as all women physiologically capable of becoming pregnant, unless they are using highly effective methods of contraception during study treatment and for 90 days after the last any dose of study treatment.

1-40. (canceled)

41. A pharmaceutical combination comprising:
i) an isolated antibody molecule capable of binding to a human Programmed Death-1 (PD-1) comprising:
(a) a heavy chain variable region (VH) comprising a HCDR1 amino acid sequence of SEQ ID NO: 1, a HCDR2 amino acid sequence of SEQ ID NO: 2, and a HCDR3 amino acid sequence of SEQ ID NO: 3; and a light chain (VL) comprising a LCDR1 amino acid sequence of SEQ ID NO: 11, a LCDR2 amino acid sequence of SEQ ID NO: 12, and a LCDR3 amino acid sequence of SEQ ID NO: 13; or
(b) a VH comprises a HCDR1 amino acid sequence of SEQ ID NO: 4, a HCDR2 amino acid sequence of SEQ ID NO: 5, and a HCDR3 amino acid sequence of SEQ ID NO: 6; and a VL comprising a LCDR1 amino acid sequence of SEQ ID NO: 14, a LCDR2 amino acid sequence of SEQ ID NO: 15, and a LCDR3 amino acid sequence of SEQ ID NO: 16; or
(c) a VH comprises a HCDR1 amino acid sequence of SEQ ID NO: 21, a HCDR2 amino acid sequence of SEQ ID NO: 22, and a HCDR3 amino acid sequence of SEQ ID NO: 23; and a VL comprising a LCDR1 amino acid sequence of SEQ ID NO: 31, a LCDR2 amino acid sequence of SEQ ID NO: 32, and a LCDR3 amino acid sequence of SEQ ID NO: 33; or
(d) a VH comprises a HCDR1 amino acid sequence of SEQ ID NO: 24, a HCDR2 amino acid sequence of SEQ ID NO: 25, and a HCDR3 amino acid sequence of SEQ ID NO: 26; and a VL comprising a LCDR1 amino acid sequence of SEQ ID NO: 34, a LCDR2 amino acid sequence of SEQ ID NO: 35, and a LCDR3 amino acid sequence of SEQ ID NO: 36;
and
ii) Compound A, which is (R,E)-N-(7-chloro-1-(1-(4-(dimethylamino)but-2-enoyl)azepan-3-yl)-1Hbenzo[d]imidazol-2-yl)-2-methylisonicotinamide, or a pharmaceutically acceptable salt thereof, preferably the mesylate salt thereof.

42. The pharmaceutical combination according to claim 41 wherein the PD-1 antibody molecule comprises a VH comprising a HCDR1 amino acid sequence of SEQ ID NO: 21, a HCDR2 amino acid sequence of SEQ ID NO: 22, and a HCDR3 amino acid sequence of SEQ ID NO: 23; and a VL comprising a LCDR1 amino acid sequence of SEQ ID NO: 31, a LCDR2 amino acid sequence of SEQ ID NO: 32, and a LCDR3 amino acid sequence of SEQ ID NO: 33; or
wherein the PD-1 antibody molecule comprises a VH comprising a HCDR1 amino acid sequence of SEQ ID NO: 24, a HCDR2 amino acid sequence of SEQ ID NO: 25, and a HCDR3 amino acid sequence of SEQ ID NO: 26; and a VL comprising a LCDR1 amino acid sequence of SEQ ID NO: 34, a LCDR2 amino acid sequence of SEQ ID NO: 35, and a LCDR3 amino acid sequence of SEQ ID NO: 36.

43. The pharmaceutical combination of claim 41, wherein the PD-1 antibody molecule comprises a heavy chain variable domain comprising an amino acid sequence at least 85%, 87%, 90%, 92%, 93%, 95%, 97% or 98% identical to any of SEQ ID NOs: 7 or 27; or preferably comprising an amino acid sequence identical to any of SEQ ID NOs: 7 or 27.

44. The pharmaceutical combination of claim 41, wherein the PD-1 antibody molecule comprises a light chain variable domain comprising an amino acid sequence at least 85%, 87%, 90%, 92%, 93%, 95%, 97% or 98% identical to any of SEQ ID NOs: 17 or 37 or preferably comprising an amino acid identical to any of SEQ ID NOs: 17 or 37.

45. The pharmaceutical combination of claim 41, wherein the PD-1antibody molecule comprises a heavy chain variable domain comprising an amino acid sequence at least 85%, 87%, 90%, 92%, 93%, 95%, 97% or 98% identical to SEQ ID NO: 27 and a light chain variable domain comprising an amino acid sequence at least 85%, 87%, 90%, 92%, 93%, 95%, 97% or 98% identical to SEQ ID NO: 37; or
wherein the PD-1antibody molecule comprises a heavy chain variable domain comprising an amino acid sequence of SEQ ID NO: 27 and a light chain variable domain comprising an amino acid sequence of SEQ ID NO: 37.

46. The pharmaceutical combination of claim 41, wherein the PD-1 antibody molecule comprises a heavy chain comprising an amino acid sequence at least 85%, 87%, 90%, 92%, 93%, 95%, 97% or 98% identical to SEQ ID NO: 29 and a light chain comprising an amino acid sequence at least 85%, 87%, 90%, 92%, 93%, 95%, 97% or 98% identical to SEQ ID NO: 39; or
wherein the PD-1 antibody molecule comprises a heavy chain comprising an amino acid sequence of SEQ ID NO: 29 and a light chain comprising an amino acid sequence of SEQ ID NO: 39.

47. The pharmaceutical combination of claim 41, wherein the PD-1 antibody molecule comprises a heavy chain variable domain comprising an amino acid sequence at least 85%, 87%, 90%, 92%, 93%, 95%, 97% or 98% identical to SEQ ID NO: 7 and a light chain variable domain comprising an amino acid sequence at least 85%, 87%, 90%, 92%, 93%, 95%, 97% or 98% identical to SEQ ID NO: 17; or
wherein the PD-1 antibody molecule comprises a heavy chain variable domain comprising an amino acid sequence of SEQ ID NO: 7 and a light chain variable domain comprising an amino acid sequence of SEQ ID NO: 17.

48. The pharmaceutical combination of claim 41, wherein the PD-1 antibody molecule comprises a heavy chain comprising an amino acid sequence at least 85%, 87%, 90%, 92%, 93%, 95%, 97% or 98% identical to SEQ ID NO: 9 and a light chain comprising an amino acid sequence at least 85%, 87%, 90%, 92%, 93%, 95%, 97% or 98% identical to SEQ ID NO: 19; or
wherein the PD-1 antibody molecule comprises a heavy chain comprising an amino acid sequence of SEQ ID NO: 9 and a light chain comprising an amino acid sequence of SEQ ID NO: 19.

49. The pharmaceutical combination of claim 41 for use in a method of treating cancer, optionally wherein the cancer is resistant or refractory to immunotherapy such as anti-PD-1 or anti-PD-L1 therapy.

50. The pharmaceutical combination for use according to claim 41, wherein the cancer is lung cancer, colorectal cancer or breast cancer.

51. The pharmaceutical combination for use according to claim 41, wherein the cancer is squamous lung cancer or NSCLC or triple-negative breast cancer (TNBC).

52. The pharmaceutical combination for use according to claim 41, wherein the anti-PD-1 antibody molecule is administered at a dose of about 300 mg to 400 mg once every three weeks or once every four weeks.

53. The pharmaceutical combination for use according to claim 41, wherein the anti-PD-1 antibody molecule is administered at a dose of about 400 mg once every four weeks; or wherein the anti-PD-1 antibody molecule is administered at a dose of about 300 mg once every three weeks.

54. The pharmaceutical combination for use according to claim 41, wherein Compound A is administered at a dose of between 5 mg and 100 mg, e.g., at a dose of 5 mg, 10 mg, 15 mg, 20 mg, 25 mg, 30 mg, 35 mg, 40 mg, 45 mg, 50 mg, 75 mg, or 100 mg.

55. The pharmaceutical combination for use according to claim 41, wherein the PD-1 antibody molecule and Compound A, or a pharmaceutically acceptable salt thereof, are administered independently at the same time or separately within time intervals, optionally followed by one or more cycles of repeat dosing; or
wherein the PD-1 antibody molecule and Compound A, or a pharmaceutically acceptable salt thereof, are administered independently at the same time or separately within time intervals, followed by one or more cycles of repeat dosing and wherein each cycle is followed by a drug holiday period.

56. The pharmaceutical combination for use according to claim 41, wherein (i) Compound A is administered in a dose of 25 mg, 50, 75 or 100 mg once a day, each day from day 1 to day 10 in a 28-day dosing cycle and the PD-1 antibody molecule is administered in a dose of 400 mg once in the same 28-day dosing cycle, optionally followed by one or more cycles of repeat dosing; or
(ii) Compound A is administered in a dose of 25 mg, 50, 75 or 100 mg once a day, each day from day 1 to day 10 in a 28-day dosing cycle and the PD-1 antibody molecule is administered in a dose of 400 mg once in the same 28-day dosing cycle, followed by one or more cycles of repeat dosing, wherein each cycle is followed by a drug holiday period.

55. An isolated antibody molecule capable of binding to a human Programmed Death-1 (PD-1) as described in claim 41, in combination with (R,E)-N-(7-chloro-1-(1-(4-(dimethylamino)but-2-enoyl)azepan-3-yl)-1Hbenzo[d]imidazol-2-yl)-2-methylisonicotinamide, or a pharmaceutically acceptable salt thereof, for use in the treatment of NSCLC, colorectal cancer, triple negative breast cancer, or a cancer that has become resistant or refractory to PD-1 or PD-L1 therapy,
optionally wherein the PD-1 or PD-L1 therapy is therapy with pembrolizumab, nivolumab, atezolizumab and MEDI4736.PD-1.


SEQUENCE LISTING (227 sequences)

SEQ ID NO: 1 - protein, 5 aa, Artificial Sequence
  source: /note=“Description of Artificial Sequence: Synthetic peptide”
TYWMH

SEQ ID NO: 2 - protein, 17 aa, Artificial Sequence
  source: /note=“Description of Artificial Sequence: Synthetic peptide”
NIYPGTGGSNFDEKFKN

SEQ ID NO: 3 - protein, 8 aa, Artificial Sequence
  source: /note=“Description of Artificial Sequence: Synthetic peptide”
WTTGTGAY

SEQ ID NO: 4 - protein, 7 aa, Artificial Sequence
  source: /note=“Description of Artificial Sequence: Synthetic peptide”
GYTFTTY

SEQ ID NO: 5 - protein, 6 aa, Artificial Sequence
  source: /note=“Description of Artificial Sequence: Synthetic peptide”
YPGTGG

SEQ ID NO: 6 - protein, 8 aa, Artificial Sequence
  source: /note=“Description of Artificial Sequence: Synthetic peptide”
WTTGTGAY

SEQ ID NO: 7 - protein, 117 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 8 - DNA, 351 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 9 - protein, 443 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - DNA, 1329 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 11 - protein, 17 aa, Artificial Sequence
  source: /note=“Description of Artificial Sequence: Synthetic peptide”
KSSQSLLDSGNQKNFLT

SEQ ID NO: 12 - protein, 7 aa, Artificial Sequence
  source: /note=“Description of Artificial Sequence: Synthetic peptide”
WASTRES

SEQ ID NO: 13 - protein, 9 aa, Artificial Sequence
  source: /note=“Description of Artificial Sequence: Synthetic peptide”
QNDYSYPYT

SEQ ID NO: 14 - protein, 13 aa, Artificial Sequence
  source: /note=“Description of Artificial Sequence: Synthetic peptide”
SQSLLDSGNQKNF

SEQ ID NO: 15 - protein, 3 aa, Artificial Sequence
  source: /note=“Description of Artificial Sequence: Synthetic peptide”
WAS

SEQ ID NO: 16 - protein, 6 aa, Artificial Sequence
  source: /note=“Description of Artificial Sequence: Synthetic peptide”
DYSYPY

SEQ ID NO: 17 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 18 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 19 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 21 - protein, 5 aa, Artificial Sequence
  source: /note=“Description of Artificial Sequence: Synthetic peptide”
TYWMH

SEQ ID NO: 22 - protein, 17 aa, Artificial Sequence
  source: /note=“Description of Artificial Sequence: Synthetic peptide”
NIYPGTGGSNFDEKFKN

SEQ ID NO: 23 - protein, 8 aa, Artificial Sequence
  source: /note=“Description of Artificial Sequence: Synthetic peptide”
WTTGTGAY

SEQ ID NO: 24 - protein, 7 aa, Artificial Sequence
  source: /note=“Description of Artificial Sequence: Synthetic peptide”
GYTFTTY

SEQ ID NO: 25 - protein, 6 aa, Artificial Sequence
  source: /note=“Description of Artificial Sequence: Synthetic peptide”
YPGTGG

SEQ ID NO: 26 - protein, 8 aa, Artificial Sequence
  source: /note=“Description of Artificial Sequence: Synthetic peptide”
WTTGTGAY

SEQ ID NO: 27 - protein, 117 aa, Artificial Sequence; identical to SEQ ID NO: 7
  source: /note=“Description of Artificial Sequence: Synthetic polypeptide”

SEQ ID NO: 28 - DNA, 351 bp, Artificial Sequence; identical to SEQ ID NO: 8
  source: /note=“Description of Artificial Sequence: Synthetic polynucleotide”

SEQ ID NO: 29 - protein, 443 aa, Artificial Sequence; identical to SEQ ID NO: 9
  source: /note=“Description of Artificial Sequence: Synthetic polypeptide”

SEQ ID NO: 30 - DNA, 1329 bp, Artificial Sequence; identical to SEQ ID NO: 10
  source: /note=“Description of Artificial Sequence: Synthetic polynucleotide”

SEQ ID NO: 31 - protein, 17 aa, Artificial Sequence
  source: /note=“Description of Artificial Sequence: Synthetic peptide”
KSSQSLLDSGNQKNFLT

SEQ ID NO: 32 - protein, 7 aa, Artificial Sequence
  source: /note=“Description of Artificial Sequence: Synthetic peptide”
WASTRES

SEQ ID NO: 33 - protein, 9 aa, Artificial Sequence
  source: /note=“Description of Artificial Sequence: Synthetic peptide”
QNDYSYPYT

SEQ ID NO: 34 - protein, 13 aa, Artificial Sequence
  source: /note=“Description of Artificial Sequence: Synthetic peptide”
SQSLLDSGNQKNF

SEQ ID NO: 35 - protein, 3 aa, Artificial Sequence
  source: /note=“Description of Artificial Sequence: Synthetic peptide”
WAS

SEQ ID NO: 36 - protein, 6 aa, Artificial Sequence
  source: /note=“Description of Artificial Sequence: Synthetic peptide”
DYSYPY

SEQ ID NO: 37 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 38 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 39 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 41 - protein, 19 aa, Artificial Sequence
  source: /note=“Description of Artificial Sequence: Synthetic peptide”
MAWVWTLPFLMAAAQSVQA

SEQ ID NO: 42 - protein, 20 aa, Artificial Sequence
  source: /note=“Description of Artificial Sequence: Synthetic peptide”
MSVLTQVLALLLLWLTGTRC

SEQ ID NO: 43 - protein, 19 aa, Artificial Sequence
  source: /note=“Description of Artificial Sequence: Synthetic peptide”
MAWVWTLPFLMAAAQSVQA

SEQ ID NO: 44 - protein, 20 aa, Artificial Sequence
  source: /note=“Description of Artificial Sequence: Synthetic peptide”
MSVLTQVLALLLLWLTGTRC

SEQ ID NO: 45-48 - skipped (4 entries)

SEQ ID NO: 49 - protein, 25 aa, Artificial Sequence [sequence omitted]

SEQ ID NO: 50 - skipped

SEQ ID NO: 51 - protein, 14 aa, Artificial Sequence
  source: /note=“Description of Artificial Sequence: Synthetic peptide”
WVRQATGQGLEWMG

SEQ ID NO: 52-54 - skipped (3 entries)

SEQ ID NO: 55 - protein, 14 aa, Artificial Sequence
  source: /note=“Description of Artificial Sequence: Synthetic peptide”
WIRQSPSRGLEWLG

SEQ ID NO: 56-57 - skipped (2 entries)

SEQ ID NO: 58 - protein, 14 aa, Artificial Sequence
  source: /note=“Description of Artificial Sequence: Synthetic peptide”
WVRQAPGQGLEWMG

SEQ ID NO: 59 - skipped

SEQ ID NO: 60 - protein, 32 aa, Artificial Sequence
  source: /note=“Description of Artificial Sequence: Synthetic polypeptide”
RVTITADKSTSTAYMELSSLRSEDTAVYYCTR

SEQ ID NO: 61-63 - skipped (3 entries)

SEQ ID NO: 64 - protein, 32 aa, Artificial Sequence
  source: /note=“Description of Artificial Sequence: Synthetic polypeptide”
RFTISRDNSKNTLYLQMNSLRAEDTAVYYCTR

SEQ ID NO: 65-66 - skipped (2 entries)

SEQ ID NO: 67 - protein, 11 aa, Artificial Sequence
  source: /note=“Description of Artificial Sequence: Synthetic peptide”
WGQGTTVTVSS

SEQ ID NO: 68-71 - skipped (4 entries)

SEQ ID NO: 72 - protein, 23 aa, Artificial Sequence
  source: /note=“Description of Artificial Sequence: Synthetic peptide”
EIVLTQSPDFQSVTPKEKVTITC

SEQ ID NO: 73-74 - skipped (2 entries)

SEQ ID NO: 75 - protein, 23 aa, Artificial Sequence
  source: /note=“Description of Artificial Sequence: Synthetic peptide”
EIVLTQSPATLSLSPGERATLSC

SEQ ID NO: 76-78 - skipped (3 entries)

SEQ ID NO: 79 - protein, 23 aa, Artificial Sequence
  source: /note=“Description of Artificial Sequence: Synthetic peptide”
DIVMTQTPLSLPVTPGEPASISC

SEQ ID NO: 80 - skipped

SEQ ID NO: 81 - protein, 23 aa, Artificial Sequence
  source: /note=“Description of Artificial Sequence: Synthetic peptide”
DVVMTQSPLSLPVTLGQPASISC

SEQ ID NO: 82 - skipped

SEQ ID NO: 83 - protein, 23 aa, Artificial Sequence
  source: /note=“Description of Artificial Sequence: Synthetic peptide”
DIQMTQSPSSLSASVGDRVTITC

SEQ ID NO: 84 - skipped

SEQ ID NO: 85 - protein, 15 aa, Artificial Sequence
  source: /note=“Description of Artificial Sequence: Synthetic peptide”
WYQQKPGQAPRLLIY

SEQ ID NO: 86-88 - skipped (3 entries)

SEQ ID NO: 89 - protein, 15 aa, Artificial Sequence
  source: /note=“Description of Artificial Sequence: Synthetic peptide”
WYQQKPGKAPKLLIY

SEQ ID NO: 90-91 - skipped (2 entries)

SEQ ID NO: 92 - protein, 15 aa, Artificial Sequence
  source: /note=“Description of Artificial Sequence: Synthetic peptide”
WYLQKPGQSPQLLIY

SEQ ID NO: 93 - skipped

SEQ ID NO: 94 - protein, 32 aa, Artificial Sequence
  source: /note=“Description of Artificial Sequence: Synthetic polypeptide”
GVPSRFSGSGSGTDFTFTISSLEAEDAATYYC

SEQ ID NO: 95-97 - skipped (3 entries)

SEQ ID NO: 98 - protein, 32 aa, Artificial Sequence
  source: /note=“Description of Artificial Sequence: Synthetic polypeptide”
GIPPRFSGSGYGTDFTLTINNIESEDAAYYFC

SEQ ID NO: 99 - skipped

SEQ ID NO: 100 - protein, 32 aa, Artificial Sequence
  source: /note=“Description of Artificial Sequence: Synthetic polypeptide”
GVPSRFSGSGSGTEFTLTISSLQPDDFATYYC

SEQ ID NO: 101-102 - skipped (2 entries)

SEQ ID NO: 103 - protein, 32 aa, Artificial Sequence
  source: /note=“Description of Artificial Sequence: Synthetic polypeptide”
GVPSRFSGSGSGTDFTFTISSLQPEDIATYYC

SEQ ID NO: 104-105 - skipped (2 entries)

SEQ ID NO: 106 - protein, 10 aa, Artificial Sequence
  source: /note=“Description of Artificial Sequence: Synthetic peptide”
FGQGTKVEIK

SEQ ID NO: 107-109 - skipped (3 entries)

SEQ ID NO: 110 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 111 - protein, 107 aa, Homo sapiens [sequence omitted]

SEQ ID NO: 112 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 113 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117-144 - skipped (28 entries)

SEQ ID NO: 145 - protein, 25 aa, Artificial Sequence
  source: /note=“Description of Artificial Sequence: Synthetic peptide”
EVQLVQSGAEVKKPGESLRISCKGS

SEQ ID NO: 146-225 - skipped (80 entries)

SEQ ID NO: 226 - DNA, 24 bp, Artificial Sequence
  source: /note=“Description of Artificial Sequence: Synthetic primer”
gctgacagactaacagactgttcc

SEQ ID NO: 227 - DNA, 18 bp, Artificial Sequence
  source: /note=“Description of Artificial Sequence: Synthetic primer”
caaatgtggtatggctga